SEQUENCE LISTING (19 sequences)

SEQ ID NO: 1 - DNA, 42 bp, synthetic construct
  source 1..42: mol_type = other DNA
tttctgttggtgctgatattgcctggtgacgcataccgaacg

SEQ ID NO: 2 - DNA, 39 bp, synthetic construct
  source 1..39: mol_type = other DNA
gaacgacatggctacgatttctgttggtgctgatattgc

SEQ ID NO: 3 - DNA, 22 bp, synthetic construct
  source 1..22: mol_type = other DNA
tttctgttggtgctgatattgc

SEQ ID NO: 4 - DNA, 37 bp, synthetic construct
  source 1..37: mol_type = other DNA
ggcggcgacctgtcgtttctgttggtgctgatattgc

SEQ ID NO: 5 - DNA, 32 bp, synthetic construct
  source 1..32: mol_type = other DNA
aaaatctagatttctgttggtgctgatattgc

SEQ ID NO: 6 - DNA, 78 bp, synthetic construct
  source 1..78: mol_type = other DNA
tatcagcacacaattgcccattatacgcgcgtataatggactattgtgtgctgatatttctgttggtgctgatattgc

SEQ ID NO: 7 - DNA, 28 bp, synthetic construct
  source 1..28: mol_type = other DNA
  modified_base 2: mod_base = OTHER; note = uracil
gtctattttctgttggtgctgatattgc

SEQ ID NO: 8 - DNA, 41 bp, synthetic construct
  source 1..41: mol_type = other DNA
  modified_base 19..25: mod_base = OTHER; note = these residues are connected by phosphorothioate bonds
ggcgtctcaaaacgcccgttttctgttggtgctgatattgc

SEQ ID NO: 9 - DNA, 39 bp, synthetic construct
  source 1..39: mol_type = other DNA
tgtgagccaaggagttgacttgcctgtcgctctatcttc

SEQ ID NO: 10 - DNA, 22 bp, synthetic construct
  source 1..22: mol_type = other DNA
acttgcctgtcgctctatcttc

SEQ ID NO: 11 - DNA, 37 bp, synthetic construct
  source 1..37: mol_type = other DNA
cgacaggtcgccgccacttgcctgtcgctctatcttc

SEQ ID NO: 12 - DNA, 32 bp, synthetic construct
  source 1..32: mol_type = other DNA
aaaaagatctacttgcctgtcgctctatcttc

SEQ ID NO: 13 - DNA, 78 bp, synthetic construct
  source 1..78: mol_type = other DNA
atagtcgtgtgttatcaggtaatatgcgcgcatattacccgttaacacacgactatacttgcctgtcgctctatcttc

SEQ ID NO: 14 - DNA, 28 bp, synthetic construct
  source 1..28: mol_type = other DNA
  modified_base 2: mod_base = OTHER; note = uracil
gtctatacttgcctgtcgctctatcttc

SEQ ID NO: 15 - DNA, 41 bp, synthetic construct
  source 1..41: mol_type = other DNA
  modified_base 19..24: mod_base = OTHER; note = the bases at positions 19-24 are connected by phosphorothioate bonds
ggcgtctcaaaacgcccgtacttgcctgtcgctctatcttc

SEQ ID NO: 16 - DNA, 34 bp, synthetic construct
  source 1..34: mol_type = other DNA
acactcggttcctcaacgaacgacatggctacga

SEQ ID NO: 17 - DNA, 60 bp, synthetic construct
  source 1..60: mol_type = other DNA
cgagacagtagaagaccatgaacaagcagcacacgataaactagacaccctactgtctcg

SEQ ID NO: 18 - DNA, 66 bp, synthetic construct
  source 1..66: mol_type = other DNA
atagaccgagacagtagaagaccatgaacaagcagcacacgataaactagacaccctactgtctcg

SEQ ID NO: 19 - DNA, 42 bp, synthetic construct
  source 1..42: mol_type = other DNA
acttgcctgtcgctctatcttcacctcggtcagattgcgagg

(19) United States
(12) Patent Application Publication
(10) Pub. No.: US 2025/0305040 A1
(43) Pub. Date: Oct. 2, 2025

(54) Methods for Producing Circular Deoxyribonucleic Acids

(71) Applicant: The Regents of the University of California, Oakland, CA (US)

(72) Inventors: John Metcalfe, San Francisco, CA (US); Jason Limberis, San Francisco, CA (US)

(21) Appl. No.: 18/698,192

(22) PCT Filed: Oct. 5, 2022
(86) PCT No.: PCT/US2022/077624

§ 371 Date: Apr. 3, 2024

Related U.S. Application Data
(60) Provisional application No. 63/252,931, filed on Oct. 6, 2021.

(51) Int. Cl. C12Q 1/686; C12Q 1/44; C12Q 1/48; C12Q 1/6806; C12Q 1/6813; C12Q 1/6869; C12Q 1/6876

(52) U.S. Cl. CPC C12Q 1/686; C12Q 1/44; C12Q 1/48; C12Q 1/6806; C12Q 1/6813; C12Q 1/6869; C12Q 1/6876; C12Y 207/07; C12Y 301/01; C12Y 605/01

(57) ABSTRACT

Provided are methods for producing circular deoxyribonucleic acids (DNAs). In some aspects, the methods include amplifying a target nucleic acid using forward and reverse primers each comprising a 5′ phosphate group, a 5′ overhang region, a 3′ hybridization region that hybridizes to the target nucleic acid, and a uracil disposed between the 5′ overhang region and the 3′ hybridization region. The amplifying produces amplicon pairs comprising phosphorylated 5′ overhangs to which adapter nucleic acids are ligated to produce a circular DNA. In other aspects, the methods comprise amplifying a target nucleic acid using forward and reverse primers each comprising first and second stem regions complementary to each other and separated by a linker region. According to such methods, the resulting amplicons are combined with an exonuclease, a DNA polymerase and a DNA ligase under conditions in which circular DNAs are produced. Related compositions and kits are also provided.

### CROSS-REFERENCE TO RELATED APPLICATIONS

This application claims the benefit of U.S. Provisional Patent Application No. 63/252,931, filed Oct. 6, 2021, which application is incorporated herein by reference in its entirety.

### STATEMENT OF GOVERNMENT SUPPORT

This invention was made with Government support under grants R01 AI131939 and U01 AI152087 awarded by the National Institutes of Health. The Government has certain rights in the invention.

### INCORPORATION BY REFERENCE OF SEQUENCE LISTING PROVIDED AS A SEQUENCE LISTING XML FILE

A Sequence Listing is provided herewith as a Sequence Listing XML, USCF-650_SEQLIST_9-19-24, created on Sep. 19, 2024 and having a size of 20,485 bytes. The contents of the Sequence Listing XML are incorporated herein by reference in their entirety.

### INTRODUCTION

A longstanding barrier to improving the treatment outcomes of many diseases (e.g., rifampin-resistant tuberculosis) is the prolonged turnaround time of culture-based drug susceptibility testing and the limited utility of most commercial molecular tests. Targeted amplicon sequencing approaches address such barriers but significant challenges remain. For example, Illumina-based methods are only capable of sequencing a maximum of 500 nucleotides; thus, to cover the regions of interest, many amplicons must be tiled across the areas of interest (some genes are >2 kb), and this requires 2 reactions per sample. Tiled reactions are problematic as they increase cost, require more initial sample thereby reducing the assays theoretical sensitivity, and make primer design and even amplification extremely complex. Moreover, Illumina-based sequencing requires high initial setup costs and large devices. Nanopore-based sequencing (e.g., using an Oxford Nanopore Tech. sequencing device) allows for long amplicon sequencing and therefore eliminates many of the issues associated with Illumina sequencing. However, the high sequencing error rate associated with nanopore-based sequencing makes clinical interpretation of non-fixed mutations complex and can lead to incorrect pretreatment being prescribed.

Rolling circle amplification (RCA) is a method for the unidirectional amplification of concatemers using a circular DNA template. Some viruses and bacteria employ RCA to amplify genomes and plasmids. In the context of sequencing, a concatemer allows for the same sequence to essentially be sequenced numerous times using a long-read sequencing platform (e.g., nanopore-based sequencing platform). These sequences can then be bioinformatically cut up and a consensus created, which substantially reduces nonsystematic sequencing error. There are, however, multiple difficulties in generating circular DNA templates RCA. These include: (1) the need to make single stranded DNA to be circularized using CircleLigase (Lucigen, WI, USA) or splint-based ligation methods, which requires exonuclease treatments and is currently a highly inefficient process; (2) the need for restriction enzyme usage and ligation into plasmids, which is also inefficient; and (3) the ligation of T-tailed hairpins to A-tailed DNA, which requires an A-tailing step and cleanup steps. The present disclosure addresses these and other shortcomings of current approaches for generating circular DNAs.

### SUMMARY

Provided are methods for producing circular deoxyribonucleic acids (DNAs). In some aspects, the methods include amplifying a target nucleic acid using forward and reverse primers each comprising a 5′ phosphate group, a 5′ overhang region, a 3′ hybridization region that hybridizes to the target nucleic acid, and a uracil disposed between the 5′ overhang region and the 3′ hybridization region. The amplifying produces amplicon pairs comprising phosphorylated 5′ overhangs to which adapter nucleic acids are ligated to produce a circular DNA. In other aspects, the methods comprise amplifying a target nucleic acid using forward and reverse primers each comprising first and second stem regions complementary to each other and separated by a linker region. According to such methods, the resulting amplicons are combined with an exonuclease, a DNA polymerase and a DNA ligase under conditions in which circular DNAs are produced. Related compositions and kits are also provided.

### BRIEF DESCRIPTION OF THE FIGURES

FIG. 1A-1B: A schematic illustration of a method for producing circular DNAs according to a first aspect of the present disclosure. A: According to this first aspect, a target nucleic acid is amplified using a forward primer and a reverse primer, wherein the forward and reverse primers each comprise a 5′ phosphate group, a 5′ overhang region, a 3′ hybridization region that hybridizes to the target nucleic acid, and a uracil disposed between the 5′ overhang region and the 3′ hybridization region. The amplifying produces amplicon pairs, each amplicon pair comprising first and second ends each comprising a phosphorylated 5′ overhang. Shown at the bottom of A is a more detailed schematic of a primer that finds use in target nucleic acid amplification according to this aspect, where “U” represents a uracil and “P” represents a 5′ phosphate group. B: Schematic illustration of the production of circular DNAs from the amplicon pairs shown in A. As shown, a 5′ phosphorylated adapter nucleic acid is ligated to the first and second ends of the amplicon pairs, wherein at each of the first and second ends of an amplicon pair, the 5′ end of the adapter nucleic acid is ligated to the 3′ end of a first strand of the amplicon pair, and the 3′ end of the adapter nucleic acid is ligated to the phosphorylated 5′ overhang of the second strand of the amplicon pair, to produce a circular DNA.

FIG. 2A-2B: A schematic illustration of a method for producing circular DNAs according to a second aspect of the present disclosure. A: According to this second aspect, a target nucleic acid is amplified using a forward primer and a reverse primer, wherein the forward and reverse primers each comprise first and second stem regions complementary to each other and separated by a linker region, and a 3′ hybridization region that hybridizes to the target nucleic acid. The amplifying produces amplicon pairs, each amplicon pair comprising first and second strands, and wherein each end of each of the first and second strands comprises the first stem region, the linker region and the second stem region. Shown at the bottom of A is a more detailed schematic of a primer pair that finds use in target nucleic acid amplification according to this aspect, where “csF” and “csR” represent stem regions having sequences complementary to each other, and “S” represents optional phosphorothioate linkages which may be included in the primers at the locations shown. B: Schematic illustration of the production of circular DNAs from the amplicon pairs shown in A by combining the amplicon pairs with an exonuclease, a DNA polymerase and a DNA ligase under conditions in which circular DNAs are produced.

FIG. 3A-3F: A schematic illustration of further details of the step shown in FIG. 2B. A: Schematic illustration of the combining of the amplicon pairs with an exonuclease, a DNA polymerase and a DNA ligase. B: Schematic illustration of the exonuclease removing the first stem region, the linker region and at least a portion of the second stem region from the 3′ end of the first strand of an amplicon pair. C: Schematic illustration of the first stem region and the second stem region of the second strand of the amplicon pair hybridizing to each other to form a stem loop structure. D: Schematic illustration of the DNA polymerase filling in a gap between the 3′ end of the first strand of the amplicon pair and the 5′ end of the second strand of the amplicon pair. E and F: Schematic illustration of the DNA ligase ligating the 3′ end of the first strand of the amplicon pair to the 5′ end of the second strand of the amplicon pair, to produce the circular DNA.

FIG. 4A-4B: A: An electrophoresis gel image showing the expected amplicons from an amplification reaction according to methods of the first aspect of the present disclosure. In this example, a Q5 PCR reaction was performed using primers containing a uracil, at ˜600 bp in relation to the NEB 100 bp DNA Ladder. The right-most lane=NTC (no template control). B: An electrophoresis gel image showing high molecular weight DNA from the successful phi29 amplification of circular generated products using short oligonucleotides. Products were generated as follows: an amplicon containing phosphorylated overhangs due to the inclusion of uracil in the primer sequence was incubated with hairpin oligonucleotides and T4 ligase with the control having no hairpin oligonucleotides added; (2) samples were then incubated with Exonuclease VIII truncated and Exonuclease I to degrade all linear DNA; (3) rolling circle amplification was then performed using phi29 polymerase; and (4) a T7 endonuclease treatment was performed to resolve branching. Lane 1=NEB 1 kb Extend DNA Ladder, Lane 5=negative control T7 endonuclease treatment, Lane 6=T7 endonuclease treatment.

FIG. 5A-5C: A: An electrophoresis gel image showing the expected amplicons from an amplification reaction according to methods of the second aspect of the present disclosure. In this example, a Q5 PCR reaction was performed using primers containing a hairpin formation sequence (as shown in FIG. 2) at ˜2.3 KB in relation to the NEB 1 kb Extend DNA Ladder. B: A bioanalyzer DNA electropherogram showing a large peak at ˜2.3 kb for the sample and a relatively minute peak for the control as expected. Samples were processed as follows: (1) an amplicon containing a sequence that forms a hairpin at s 50° C. was digested with mixture of T7 exonuclease, DNA ligase and DNA polymerase, resulting in the formation of circular DNA; and (2) the circular DNA was incubated with Exonuclease VIII truncated and Exonuclease I to degrade all linear DNA, showing that the circularization was successful (red line). C: RCA reactions using the generated circular DNA as template, Phi29 polymerase, and different combinations of short oligonucleotides (lanes 2, 3 and 5) and random hexamers (lane 4) showing large (≥48.5 kb) DNA fragments and large (likely) hyperbranched fragments in the wells. Lane 1=NEB 1 kb Extend DNA Ladder.

FIG. 6A-6E: Schematic illustration of methods for the circularization of PCR amplicons.

FIG. 7: Methods for circularizing a ˜700 bp high GC amplicon. Successful circularization was achieved for self-circularization of ssDNA using CircleLigase, using splints and T4 ligase, Ampligase, and Taq DNA ligase; DumBell ligation using T4 ligase, uDumBell ligation and easyBD ligation. The results panel shows the TapeStation gel image for the control (C) and sample (S) which were treated with specific exonucleases to remove all noncircular or non-pseudocircular DNA; the green arrow indicates the expected band location. Circularized products using a Gibson cloning-based approach or a restriction enzyme approach using T4 ligase, Ampligase, or Taq DNA ligase, could not be generated. The Kinase ligase approach did not form a single amplicon circular DNA, but large circular concatemers were detected (not visible).

FIG. 8: Structure of an example uDumBell adapter sequence that forms a stable hairpin at temperatures≤37° C.

FIG. 9A-C: Schematic illustration of methods for the circularization of PCR amplicons and the joining of amplicons with applications in cloning.

FIG. 10: Structure of the single-stranded splint oligonucleotide used in the splint method, bound to the single-stranded amplicon (SEQ ID NO:16).

FIG. 11: Structure of the tail sequence of the easyDB method that forms a hairpin at temperature<55° C., but not ≥55° C.

### DETAILED DESCRIPTION

Before the methods, compositions and kits of the present disclosure are described in greater detail, it is to be understood that the methods, compositions and kits are not limited to particular embodiments described, as such may, of course, vary. It is also to be understood that the terminology used herein is for the purpose of describing particular embodiments only, and is not intended to be limiting, since the scope of the methods, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, compositions and kits. The upper and lower limits of these smaller ranges may independently be included in the smaller ranges and are also encompassed within the methods, compositions and kits, subject to any specifically excluded limit in the stated range. Where the stated range includes one or both of the limits, ranges excluding either or both of those included limits are also included in the methods, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, compositions and kits belong. Although any methods, compositions and kits similar or equivalent to those described herein can also be used in the practice or testing of the methods, compositions and kits, representative illustrative methods, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, compositions and kits, which are, for clarity, described in the context of separate embodiments, may also be provided in combination in a single embodiment. Conversely, various features of the methods, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

The present disclosure provides methods for producing circular deoxyribonucleic acids (DNAs). Existing approaches for producing circularized DNAs include using CircleLigase (Lucigen, WI, USA) or splint-based ligation methods to circularize single stranded DNA, which methods require exonuclease treatments and are highly inefficient processes. Existing approaches also include the ligation of T-tailed hairpins to A-tailed DNA, which requires an A-tailing step and cleanup steps. As will be appreciated upon review of the present disclosure, the circularization methods of the present disclosure constitute a substantial improvement with respect to efficiency and other factors compared to the existing approaches for circularizing nucleic acids. Circularized DNAs produced according to the methods of the present disclosure find use in a variety of contexts. By way of example, the circularized DNAs find use as templates for rolling circle amplification, e.g., for producing concatemers for downstream analysis. Downstream analyses of interest include, but are not limited to, nanopore-based or other next generation sequence analysis, where the concatemers enable redundant sequencing of target sequences for improved sequencing accuracy. As a further example, RCA may be coupled with detectable probes to create detection assays for genes and/or pathogens of interest. Also by way of example, the circularization methods of the present disclosure find use in rapid cloning of sequences of interest, where the primers and/or any other oligonucleotides employed in the methods may be designed such that the resulting DNAs contain overhangs complementary to a plasmid (vector) to which they will be ligated and that contain elements for replication in a host cell of interest, e.g., a prokaryotic and/or eukaryotic host cell. Details of aspects and embodiments of the methods of the present disclosure will now be provided.

According to a first aspect of the present disclosure, provided are methods for producing circular DNAs, the methods comprising amplifying a target nucleic acid using a forward primer and a reverse primer, wherein the forward and reverse primers each comprise a 5′ phosphate group, a 5′ overhang region, a 3′ hybridization region that hybridizes to the target nucleic acid, and a uracil disposed between the 5′ overhang region and the 3′ hybridization region. The amplifying produces amplicon pairs, each amplicon pair comprising first and second ends each comprising a phosphorylated 5′ overhang.

As will be appreciated with the benefit of the present disclosure, the design of the primers to incorporate a uracil between the 5′ overhang region and the 3′ hybridization region, where the uracil halts the polymerase during amplification, is responsible for the presence of the phosphorylated 5′ overhang at each end of each amplicon pair. The uracil therefore obviates the need to add or remove bases at the ends of the amplicons (and the associated clean up steps) to facilitate circularization.

The methods according to the first aspect further comprise ligating a 5′ phosphorylated adapter nucleic acid to the first and second ends of the amplicon pairs, wherein at each of the first and second ends of an amplicon pair, the 5′ end of the adapter nucleic acid is ligated to the 3′ end of a first strand of the amplicon pair, and the 3′ end of the adapter nucleic acid is ligated to the phosphorylated 5′ overhang of the second strand of the amplicon pair, to produce a circular DNA.

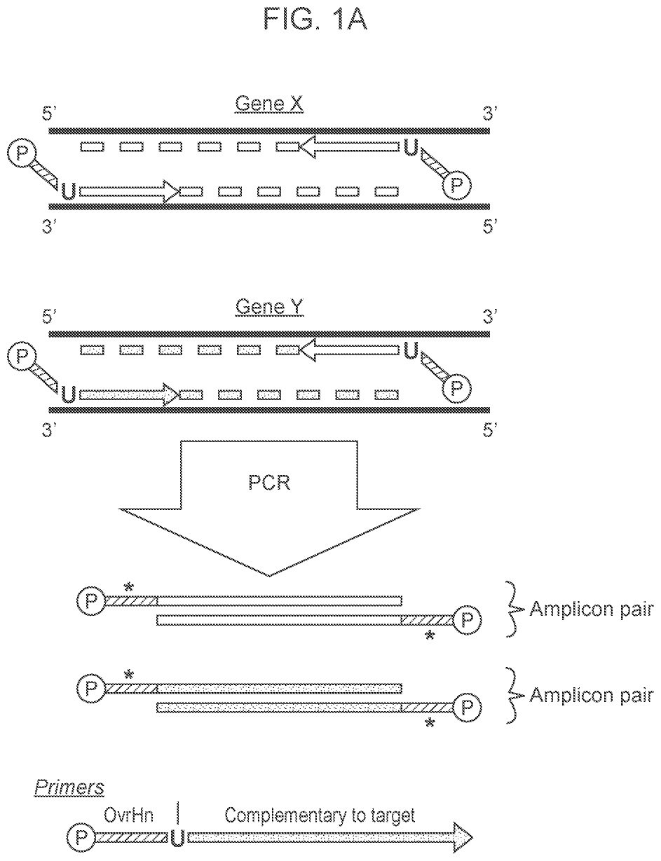

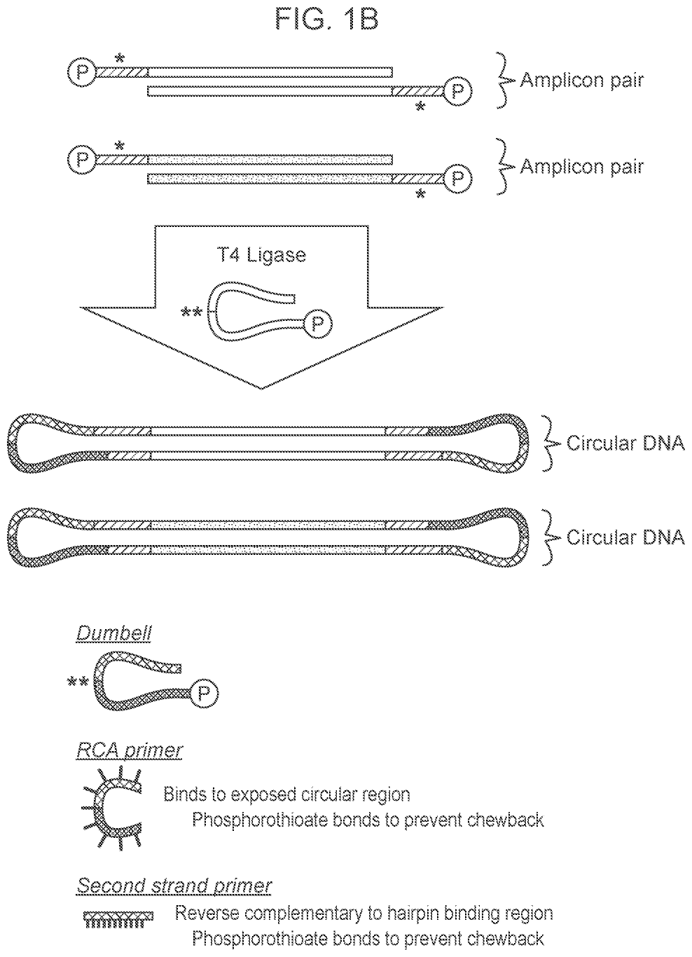

A non-limiting example of a circularization method according to the first aspect is schematically illustrated in FIG. 1. As shown in FIG. 1A, target sequences of first and second target nucleic acids (designated “Gene X” and “Gene Y”) are amplified by PCR using forward and reverse primers each comprising a 5′ phosphate group, a 5′ overhang region (“OvrHn”), a 3′ hybridization region that hybridizes (by virtue of complementarity) to the target nucleic acid, and a uracil (“U”) disposed between the 5′ overhang region and the 3′ hybridization region. The resulting amplicon pairs each comprise first and second ends, the first and second ends each comprising a phosphorylated 5′ overhang (indicated by asterisks (*) in FIG. 1).

Now with reference to FIG. 1B, a 5′ phosphorylated adapter nucleic acid (indicated by double asterisks (**) and referred to as “Dumbell” in FIG. 1B) is ligated to the first and second ends of the amplicon pairs, wherein at each of the first and second ends of an amplicon pair, the 5′ end of the adapter nucleic acid is ligated to the 3′ end of a first strand of the amplicon pair, and the 3′ end of the adapter nucleic acid is ligated to the phosphorylated 5′ overhang of the second strand of the amplicon pair, to produce a circular DNA. In some embodiments, the resulting circular DNAs are used as templates for RCA. A non-limiting example of a primer which finds use for performing the RCA is shown at the bottom of FIG. 1B (designated “RCA primer”). As shown, this primer is complementary to and anneals to the exposed loop (or “circular”) region of the circular DNA. Also shown is a second strand primer that may be employed according to some embodiments. In some embodiments, any of the RCA and/or second strand primers may include phosphorothioate linkages to prevent degradation (or “chewback”) by an exonuclease employed at one or more steps of the methods.

According to a second aspect of the present disclosure, provided are methods for producing circular DNAs, the methods comprising amplifying a target nucleic acid using a forward primer and a reverse primer, wherein the forward and reverse primers each comprise first and second stem regions complementary to each other and separated by a linker region, and a 3′ hybridization region that hybridizes to the target nucleic acid. In some embodiments, the forward primer, the reverse primer, or both, comprise one or more exonuclease resistance features (e.g., phosphorothioate linkages) present within the second stem region, the 3′ hybridization region, or both. The amplifying produces amplicon pairs, each amplicon pair comprising first and second strands, and wherein each end of each of the first and second strands comprises the first stem region, the linker region and the second stem region. The methods according to the second aspect further comprise combining the amplicon pairs with an exonuclease, a DNA polymerase and a DNA ligase (e.g., in a single reaction mixture, e.g., in a single tube, vial, well or the like) under conditions in which, at each end of the amplicon pairs: the exonuclease removes the first stem region, the linker region and at least a portion of the second stem region from the 3′ end of the first strand of an amplicon pair; the first stem region and the second stem region of the second strand of the amplicon pair hybridize to each other to form a stem loop structure; the DNA polymerase fills in a gap between the 3′ end of the first strand of the amplicon pair and the 5′ end of the second strand of the amplicon pair; and the DNA ligase ligates the 3′ end of the first strand of the amplicon pair to the 5′ end of the second strand of the amplicon pair, to produce a circular DNA.

According to some embodiments, the complementary sequences of the first stem region and the second stem region are designed to hybridize at a particular temperature or desired range of temperatures, such that the conditions may comprise bringing the temperature of the reaction mixture to the particular temperature or with the desired range of temperatures at which the stem regions hybridize/anneal (e.g., specifically) to form the stem loop structure. In some embodiments, the complementary sequences of the first stem region and the second stem region are designed to hybridize/anneal (e.g., specifically) to form the stem loop structure at a temperature from 45° C. to 55° C. (e.g., about 50° C.), and the conditions comprise bringing the temperature of the reaction mixture to from 45° C. to 55° C. (e.g., about 50° C.).

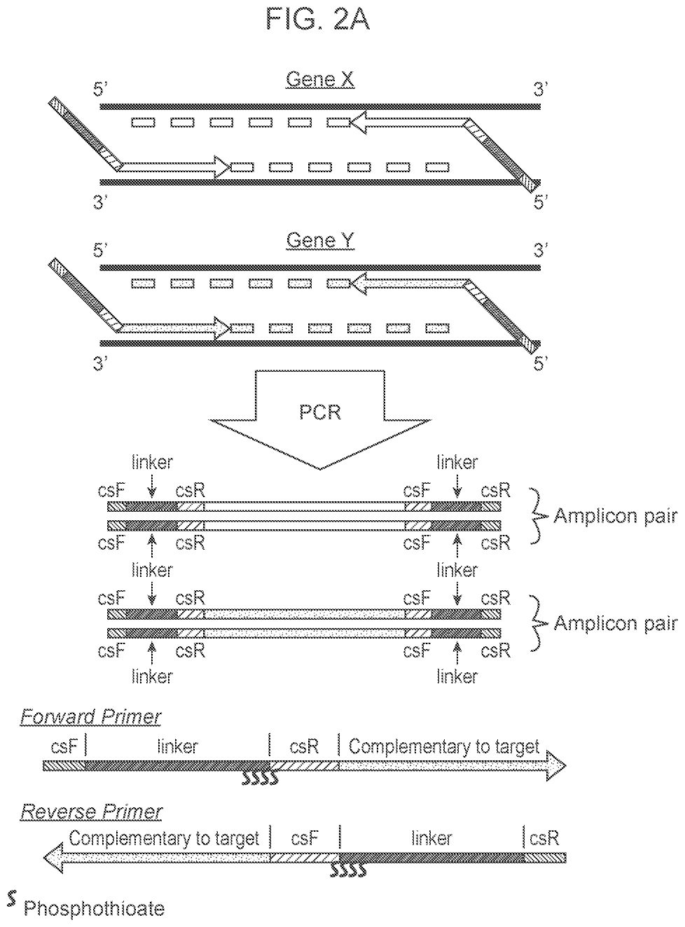

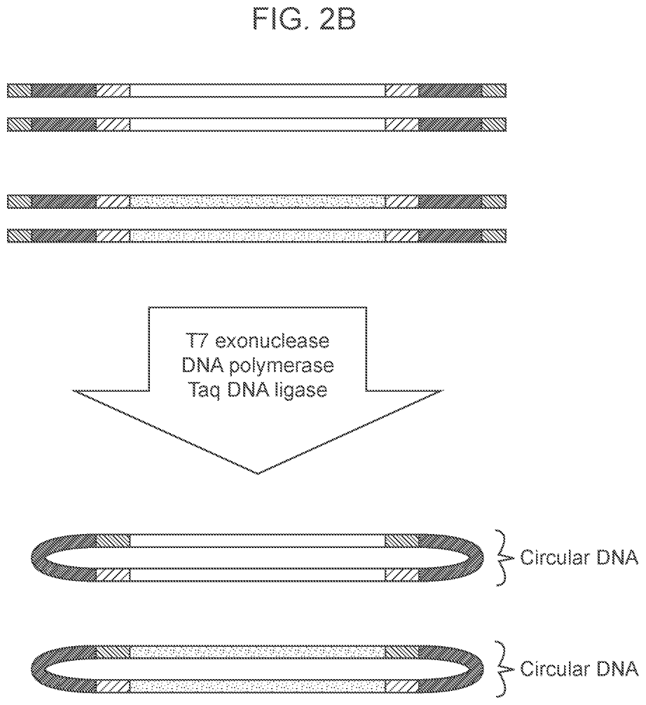

A non-limiting example of a circularization method according to the second aspect is schematically illustrated in FIG. 2. As shown in FIG. 2A, target sequences of first and second target nucleic acids (designated “Gene X” and “Gene Y”) are amplified by PCR using forward and reverse primers each comprising first and second stem regions complementary to each other (designated “csF” and “csR” in the forward and reverse primers schematically illustrated at the bottom of FIG. 2A) separated by a linker region, and a 3′ hybridization region (“Complementary to target”) that hybridizes to the target nucleic acid. The amplifying produces amplicon pairs, each amplicon pair comprising first and second strands, and wherein each end of each of the first and second strands comprises the first stem region, the linker region and the second stem region.

Now with reference to FIG. 2B, the amplicon pairs are combined with an exonuclease, a DNA polymerase and a DNA ligase, e.g., in a single reaction mixture, e.g., in a single tube, vial, well or the like. In the particular example shown in FIG. 2, the exonuclease is a T7 exonuclease, the polymerase is a DNA polymerase, and the DNA ligase is a Taq DNA ligase. The combining is under conditions in which a circular DNA is produced as set forth above and below.

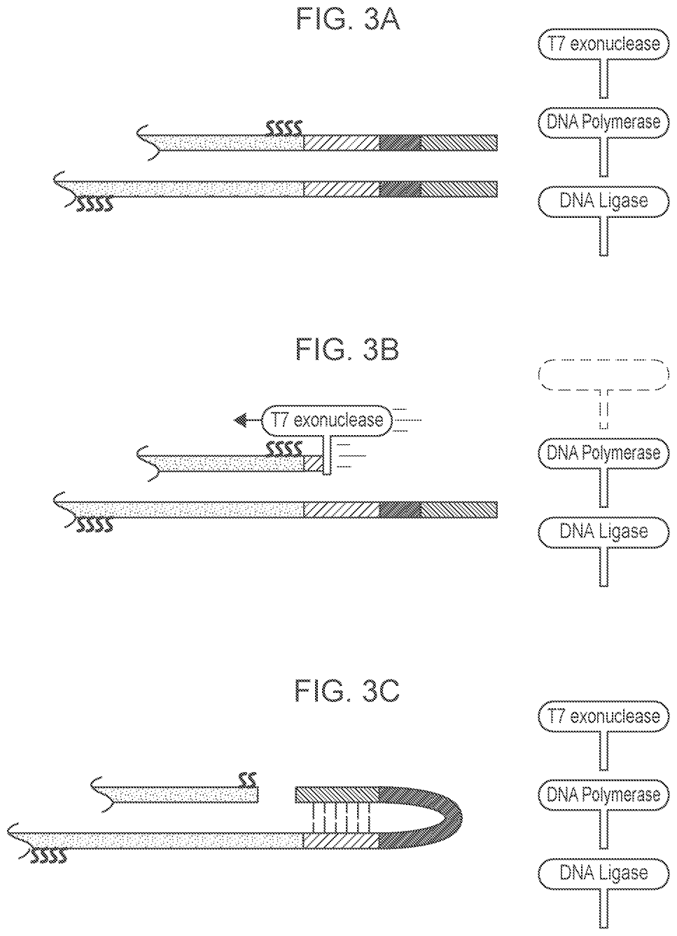

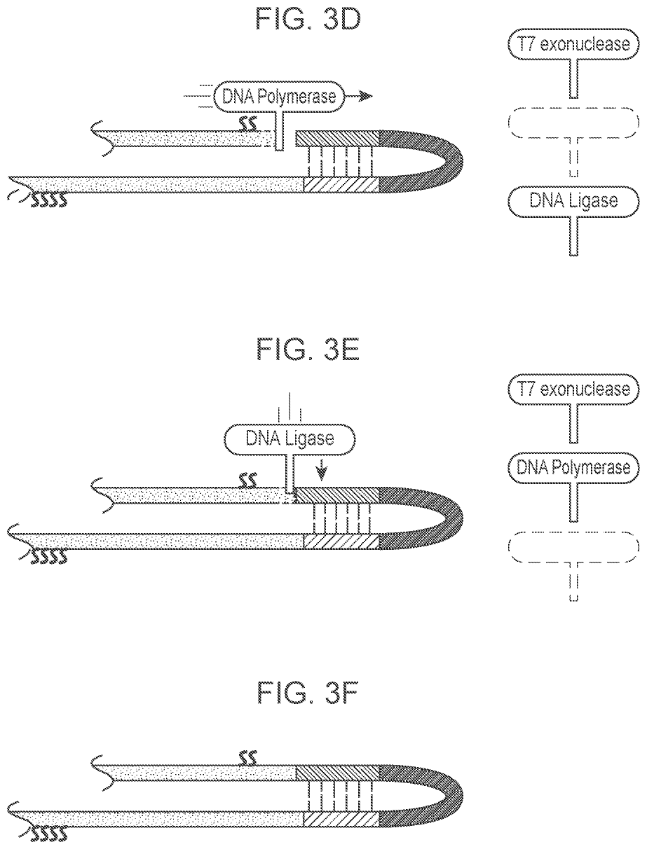

Shown in FIG. 3 is a schematic illustration of further details of the combining/circularization step according to the methods of the second aspect. FIG. 3A schematically illustrates the combining of the amplicon pairs with an exonuclease, a DNA polymerase and a DNA ligase. FIG. 3B schematically illustrates the exonuclease removing the first stem region, the linker region and at least a portion of the second stem region from the 3′ end of the first strand of an amplicon pair. FIG. 3C schematically illustrates the first stem region and the second stem region of the second strand of the amplicon pair hybridizing to each other to form a stem loop structure. FIG. 3D schematically illustrates the DNA polymerase filling in a gap between the 3′ end of the first strand of the amplicon pair and the 5′ end of the second strand of the amplicon pair. FIG. 3E and FIG. 3F schematically illustrate the DNA ligase ligating the 3′ end of the first strand of the amplicon pair to the 5′ end of the second strand of the amplicon pair, and the resulting circular DNA, respectively.

According to any of the first and second aspects of the methods of the present disclosure, the target nucleic acid may be any target nucleic acid of interest. In certain embodiments, the target nucleic acid is a deoxyribonucleic acid (DNA), non-limiting examples of which include genomic DNA (e.g., a gene, an intergenic region, a polymorphic region, and/or the like), complementary DNA (or “cDNA”, synthesized from any RNA or DNA of interest), recombinant DNA (e.g., plasmid DNA), circulating tumor DNA (ctDNA) (e.g., isolated from a liquid biopsy), cell-free DNA (cfDNA) (e.g., isolated from blood or a fraction thereof), and any other DNAs of interest.

According to some embodiments, the target nucleic acid is one present in a cell-free nucleic acid sample, e.g., cell-free DNA, cell-free RNA, or both. Such cell-free nucleic acids may be obtained from any suitable source. In certain embodiments, the cell-free nucleic acids are from a body fluid sample selected from the group consisting of: whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. In certain embodiments, the cell-free nucleic acids are cell-free fetal DNAs. According to some embodiments, the cell-free nucleic acids are circulating tumor DNAs. In certain embodiments, the cell-free nucleic acids comprise infectious agent DNAs. According to some embodiments, the cell-free nucleic acids comprise DNAs from a transplant.

The term “cell-free nucleic acid” as used herein can refer to nucleic acid isolated from a source having substantially no cells. Cell-free nucleic acid may be referred to as “extracellular” nucleic acid, “circulating cell-free” nucleic acid (e.g., CCF fragments, ccf DNA) and/or “cell-free circulating” nucleic acid. Cell-free nucleic acid can be present in and obtained from blood (e.g., from the blood of an animal, from the blood of a human subject). Cell-free nucleic acid often includes no detectable cells and may contain cellular elements or cellular remnants. Non-limiting examples of acellular sources for cell-free nucleic acid are described above. Obtaining cell-free nucleic acid may include obtaining a sample directly (e.g., collecting a sample, e.g., a test sample) or obtaining a sample from another who has collected a sample. Without being limited by theory, cell-free nucleic acid may be a product of cell apoptosis and cell breakdown, which provides basis for cell-free nucleic acid often having a series of lengths across a spectrum (e.g., a “ladder”). In some embodiments, sample nucleic acid from a test subject is circulating cell-free nucleic acid. In some embodiments, circulating cell free nucleic acid is from blood plasma or blood serum from a test subject. In some embodiments, the cell-free nucleic acid is degraded.

Cell-free nucleic acid can include different nucleic acid species, and therefore is referred to herein as “heterogeneous” in certain embodiments. For example, a sample from a subject having cancer can include nucleic acid from cancer cells (e.g., tumor, neoplasia) and nucleic acid from non-cancer cells. In another example, a sample from a pregnant female can include maternal nucleic acid and fetal nucleic acid. In another example, a sample from a subject having an infection or infectious disease can include host nucleic acid and nucleic acid from the infectious agent (e.g., bacteria, fungus, protozoa). In another example, a sample from a subject having received a transplant can include host nucleic acid and nucleic acid from the donor organ or tissue. In some instances, cancer, fetal, infectious agent, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, fetal, infectious agent, or transplant nucleic acid). In another example, heterogeneous cell-free nucleic acid may include nucleic acid from two or more subjects (e.g., a sample from a crime scene).

According to any of the embodiments of the present disclosure, the target nucleic acid may be one isolated from a tumor nucleic acid sample (that is, a nucleic acid sample isolated from a tumor). “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like.

According to some embodiments, the target nucleic acid is a target ribonucleic acid (RNA), e.g., where the amplification step may comprise reverse transcription PCR (RT-PCR) or the like. A target RNA may be any type of RNA (or sub-type thereof) including, but not limited to, a messenger RNA (mRNA), a microRNA (miRNA), a small interfering RNA (siRNA), a transacting small interfering RNA (ta-siRNA), a natural small interfering RNA (nat-siRNA), a ribosomal RNA (rRNA), a transfer RNA (tRNA), a small nucleolar RNA (snoRNA), a small nuclear RNA (snRNA), a long non-coding RNA (lncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared siRNA (esiRNA), a small temporal RNA (stRNA), a signal recognition RNA, a telomere RNA, a ribozyme, or any combination of RNA types thereof or subtypes thereof.

According to some embodiments, the target nucleic acid sample is one present in an environmental nucleic acid sample. In certain embodiments, the environmental nucleic acid sample is a liquid environmental nucleic acid sample. The liquid environmental sample may be, e.g., drinking (or potable) water, surface water (e.g., river water, stream water, lake water, reservoir water, wetland water, bog water, or the like), ground water, waste water, well water, water from an unsaturated zone, rain water, run-off water, sea water, liquid industrial waste, sewage, surface films, or the like. In certain embodiments, the environmental nucleic acid sample is a solid environmental nucleic acid sample. The solid environmental sample may be from, e.g., ice, snow, soil, sewage sludge, bottom sediments, dust from electrofilters, vacuuming dust, plant material, forest floor, industrial waste, municipal waste, ashes, or the like.

In certain embodiments, the target nucleic acid is pathogen DNA and/or RNA. Pathogens of interest include, but are not limited to, viral pathogens, bacterial pathogens, amoebic pathogens, parasitic pathogens, and fungal pathogens. According to some embodiments, the target nucleic acid is isolated from an infected host comprising the pathogen DNA and/or RNA. Infected hosts of interest include, but are not limited to, a terrestrial animal, a human, a terrestrial plant, an aquatic animal, and an aquatic plant. By “terrestrial” is meant an animal or plant that lives primarily on land (e.g., at least 75% of the time) as opposed to living in water. By “aquatic” is meant an animal or plant that lives primarily in water (e.g., at least 75% of the time) as opposed to on land. According to some embodiments, the DNA and/or RNA is isolated from excreta (e.g., urine and/or feces) of the infected host. In certain embodiments, the DNA and/or RNA is isolated from material shed from the infected host, non-limiting examples of which include hair and/or skin. Methods involving pathogen DNA and/or RNA and infected hosts may further comprise distinguishing the pathogen DNA and/or RNA from the infected host's DNA and/or RNA. Such methods may further include, subsequent to the distinguishing, analyzing the pathogen DNA and/or RNA, e.g., by sequencing as described in detail elsewhere herein.

The target nucleic acid may be present in any nucleic acid sample of interest, including but not limited to, a nucleic acid sample isolated from a single cell, a plurality of cells (e.g., cultured cells), a tissue, an organ, or an organism (e.g., bacteria, yeast, or the like). In certain embodiments, the nucleic acid sample is isolated from a cell(s), tissue, organ, and/or the like of a mammal (e.g., a human, a rodent (e.g., a mouse), or any other mammal of interest). According to some embodiments, the nucleic acid sample is isolated from a source other than a mammal, such as bacteria, yeast, insects (e.g., drosophila), amphibians (e.g., frogs (e.g., Xenopus)), viruses, plants, or any other non-mammalian nucleic acid sample source.

Approaches, reagents and kits for isolating, purifying and/or concentrating DNA and RNA from sources of interest are known in the art and commercially available. For example, kits for isolating DNA from a source of interest include the DNeasy®, RNeasy®, QIAamp®, QIAprep® and QIAquick® nucleic acid isolation/purification kits by Qiagen, Inc. (Germantown, Md); the DNAzol®, ChargeSwitch®, Purelink®, GeneCatcher® nucleic acid isolation/purification kits by Life Technologies, Inc. (Carlsbad, CA); the NucleoMag®, NucleoSpin®, and NucleoBond® nucleic acid isolation/purification kits by Clontech Laboratories, Inc. (Mountain View, CA). In certain embodiments, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

According to any of the first and second aspects and embodiments thereof of the methods of the present disclosure, the methods may further comprise performing rolling circle amplification (RCA) using the produced circular DNAs as templates, wherein the RCA produces concatemers comprising repeating segments each comprising a target nucleic acid sequence. The target nucleic acid sequence corresponds to the sequence of the target nucleic acid amplified during the amplifying step. As used herein, the term “rolling circle amplification” or “RCA” refers to an amplification (e.g., isothermal amplification) that generates linear concatemerized copies of a circular nucleic acid template using a strand-displacing polymerase. During RCA, the polymerase continuously adds single nucleotides to a primer (e.g., an oligonucleotide primer or a primer produced by nicking a double-stranded circular DNA (e.g., using an endonuclease)) annealed to the circular template which results in a concatemeric single-stranded DNA (ssDNA) that contains tandem repeats (or “linked units”) (e.g., tens, hundreds, thousands, or more tandem repeats) complementary to the circular template. Suitable strand-displacing polymerases that may be employed include, but are not limited to, Phi29 polymerase, Bst polymerase, Vent exo-DNA polymerase, and the like. Reagents, protocols and kits for performing RCA are known and include, e.g., the RCA DNA Amplification Kit available from Molecular Cloning Laboratories; and TruePrime™ RCA Kit available from Expedeon.

Any suitable primer(s) may be employed when performing RCA using the produced circular DNAs as templates. In certain embodiments, when the circular DNAs include loop regions (sometimes referred to herein as an “exposed circular regions”), the RCA is performed using an RCA primer that hybridizes to a loop region of the circular DNAs. According to some embodiments, when the circular DNAs were produced according to the methods of the first aspect, the RCA uses a primer that anneals to the adapter nucleic acid. In certain embodiments, when the circular DNAs were produced according to the methods of the second aspect, the RCA uses a primer that anneals to the loop portion of the stem loop structure. According to some embodiments, the methods further comprise performing second strand synthesis using a second strand primer and the RCA products as templates.

As used herein, an “oligonucleotide” is a single-stranded multimer of nucleotides from 5 to 500 nucleotides, e.g., 5 to 100 nucleotides. Oligonucleotides may be synthetic or may be made enzymatically, and, in some embodiments, are 5 to 50 nucleotides in length. Oligonucleotides may contain ribonucleotide monomers (i.e., may be oligoribonucleotides or “RNA oligonucleotides”), deoxyribonucleotide monomers (i.e., may be oligodeoxyribonucleotides or “DNA oligonucleotides”), or a combination thereof. Oligonucleotides may be 10 to 20, 20 to 30, 30 to 40, 40 to 50, 50 to 60, 60 to 70, 70 to 80, 80 to 100, 100 to 150 or150 to 200, or up to 500 nucleotides in length, for example.

Amplification primers, RCA primers, second strand synthesis primers, and the like are selected and/or designed such that they are complementary to their intended targets. The term “complementary” as used herein refers to a nucleotide sequence that base-pairs by non-covalent bonds to all or a region of a target nucleic acid (e.g., a region of the template nucleic acid, a region of the product nucleic acid, or the like). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%). The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

For amplification of target nucleic acids, RCA, second strand synthesis, and/or the like, the methods comprise annealing the relevant primer(s) to the target(s) under hybridization conditions. As used herein, the term “hybridization conditions” means conditions in which a primer specifically hybridizes to a region of the target (e.g., target nucleic acid, circular DNA, concatemer, or the like). Whether a primer specifically hybridizes to a target is determined by such factors as the degree of complementarity between the primer and the target and the temperature at which the hybridization occurs, which may be informed by the melting temperature (T_{M}) of the primer. The melting temperature refers to the temperature at which half of the primer-target duplexes remain hybridized and half of the duplexes dissociate into single strands. The Tm of a duplex may be experimentally determined or predicted using the following formula Tm=81.5+16.6(log 10[Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

Any target nucleic acid amplification, RCA, and/or second strand synthesis primers employed when performing the methods of the present disclosure may include one or more nucleotides (or analogs thereof) that are modified or otherwise non-naturally occurring. For example, a primer may include one or more nucleotide analogs (e.g., LNA, FANA, 2′-O-Me RNA, 2′-fluoro RNA, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to a primer during target nucleic acid amplification, RCA, and/or second strand synthesis. In certain embodiments, one or both primers for target nucleic acid amplification, an RCA primer, and/or second strand synthesis primer may comprise one or more exonuclease resistance features, e.g., when it is desirable to prevent “chewback” by an exonuclease that may be employed when performing the methods. A non-limiting example of an exonuclease resistance feature that may be incorporated into a desired region of any of the primers employed is one or more phosphorothioate linkages. By way of example, when performing the methods according to the second aspect of the present disclosure, the forward primer, the reverse primer, or both, comprise one or more exonuclease resistance features present within the second stem region, the 3′ hybridization region, or both.

In certain embodiments, when the methods comprise performing RCA using the produced circular DNAs as templates, the methods may further comprise sequencing the concatemers using a nanopore sequencing device. According to some embodiments, sequencing the concatemers using a nanopore sequencing device comprises applying a potential difference across a nanopore, and detecting (e.g., monitoring) electrical signals from the nanopore while exposing a concatemer to the nanopore in a sequential manner. In certain embodiments, exposing the concatemer to the nanopore in a sequential manner includes translocating at least a portion of the concatemer through the nanopore.

Any nanopore device/apparatus suitable for exposing the concatemer to a nanopore (e.g., translocating the concatemer through a nanopore) and detecting/monitoring ionic current through the nanopore during the exposing/translocating may be employed when practicing the subject methods. For example, a suitable nanopore device may include a chamber including an aqueous solution and a membrane that separates the chamber into two sections, the membrane including a nanopore formed therein. Electrical measurements may be made using single channel recording equipment such as that described, e.g., in Lieberman et al. (2010) J. Am. Chem. Soc. 132(50):17961-72; Stoddart et al. (2009) PNAS 106(19):7702-7; U.S. Pat. No. 9,481,908; and U.S. Patent Application Publication No. US2014/0051068; the disclosures of which are incorporated herein by reference in their entireties for all purposes. Alternatively, electrical measurements may be made using a multi-channel system, for example as described in U.S. Patent Application Publication No. US2015346149, the disclosure of which is incorporated herein by reference in its entirety for all purposes.

In nanopore sequencing, the nanopore serves as a biosensor and provides the sole passage through which an ionic solution on the cis side of the membrane contacts the ionic solution on the trans side. A constant voltage bias (trans side positive) produces an ionic current through the nanopore and drives ssDNA or ssRNA in the cis chamber through the pore to the trans chamber. A processive enzyme (e.g., a helicase, polymerase, nuclease, or the like) may be bound to the polynucleotide such that its step-wise movement controls and ratchets the nucleotides through the small-diameter nanopore, nucleobase by nucleobase. Because the ionic conductivity through the nanopore is sensitive to the presence of the nucleobase's mass and its associated electrical field, the ionic current levels through the nanopore reveal the sequence of nucleobases in the translocating strand. A patch clamp, a voltage clamp, or the like, may be employed.

Suitable conditions for measuring ionic currents through transmembrane pores (e.g., protein pores, solid state pores, etc.) are known in the art. Typically, a voltage is applied across the membrane and pore. The voltage used may be from +2 V to −2 V, e.g., from −400 mV to +400 mV. The voltage used may be in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage may be in the range of from 100 mV to 240 mV, e.g., from 120 mV to 220 mV.

The methods are typically carried out in the presence of a suitable charge carrier, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or I-ethyl-3-methyl imidazolium chloride. Generally, the salt is present in the aqueous solution in the chamber. Potassium chloride (KCl), sodium chloride (NaCl) or cesium chloride (CsCl) may be used, for example. The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M, or from 1 M to 1.4 M. The salt concentration may be from 150 mM to 1 M. The methods are preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

In some embodiments, the rate at which the concatemer is exposed to the nanopore is controlled using a processive enzyme. Non-limiting examples of processive enzymes that may be employed include polymerases (e.g., a phi29 or other suitable polymerase) and helicases, e.g., a Hel308 helicase, a RecD helicase, a Tral helicase, a Tral subgroup helicase, an XPD helicase, or the like. The concatemer may be bound by the processive enzyme (e.g., by binding of the processive enzyme to a recognition site present in a sequencing adapter located at an end of the concatemer), followed by the resulting complex being drawn to the nanopore, e.g., by a potential difference applied across the nanopore. In other aspects, the processive enzyme may be located at the nanopore (e.g., attached to or adjacent to the nanopore) such that the processive enzyme binds the concatemer upon arrival of the concatemer at the nanopore.

The nanopore may be present in a solid-state film, a biological membrane, or the like. In some embodiments, the nanopore is a solid-state nanopore. In other embodiments, the nanopore is a biological nanopore. The biological nanopore may be, e.g., an alpha-hemolysin-based nanopore, a Mycobacterium smegmatis porin A (MspA)-based nanopore, or the like.

Details for obtaining raw sequencing reads of nucleic acid molecules of interest using nanopores are described, e.g., in Feng et al. (2015) Genomics, Proteomics & Bioinformatics 13(1):4-16. Raw sequencing reads may be obtained using, e.g., a MinION™, GridIONx5™, PromethION™, or SmidgION™ nanopore-based sequencing system, available from Oxford Nanopore Technologies. Detailed design considerations and protocols for carrying out nanopore-based sequencing are provided with such systems.

Once a raw sequencing read of the concatemer is obtained, the present methods further include identifying the repeating segments in the raw sequencing read. In some embodiments, identifying the repeating segments in the raw sequencing read includes identifying at least one sequence of the known heterologous sequence in the raw sequencing read. In certain aspects, the at least one sequence of the known heterologous sequence is identified in the raw sequencing read using a BLAST-Like Alignment Tool (BLAT).

In some embodiments, identifying the repeating segments in the raw sequencing read includes subjecting the raw sequencing read to a modified Smith-Waterman self-to-self alignment. The Smith-Waterman algorithm is a dynamic programming algorithm that performs local sequence alignment for determining similar regions between two strings of nucleic acid or protein sequences. Instead of looking at the entire sequence, the Smith-Waterman algorithm compares segments of all possible lengths and optimizes the similarity measure. In certain aspects, identifying the repeating segments in the raw sequencing read comprises parsing a score matrix of the modified Smith-Waterman self-to-self alignment.

According to some embodiments, the methods of the present disclosure comprise producing a consensus sequence of the target nucleic acid sequence. In certain embodiments, the consensus sequence is produced by combining the sequences of the repeating segments using a partial order alignment (POA).

Once a consensus sequence of the target nucleic acid sequence is produced, the methods of the present disclosure may further include subjecting the consensus sequence to error-correction. In some embodiments, subjecting the consensus sequence to error-correction comprises subjecting the consensus sequence to rapid consensus (Racon).

The methods of the present disclosure find use in a variety of contexts including research, clinical (e.g., clinical diagnostic), forensic, and other contexts. In certain embodiments, the methods may be employed for sample analysis (e.g., for clinical diagnostic purposes) and incorporated into the workflow, reagents, consumables (e.g., cartridges), and/or the like of a sample analysis system of interest. Non-limiting examples of commercially available sample analysis systems in which the methods and compositions of the present disclosure find use is the GeneXpert® family of sample analysis systems available from Cepheid (Sunnyvale, CA).

### Compositions

Aspects of the present disclosure further include compositions. A composition of the present disclosure may include any of the reagents (e.g., nucleic acids, primers, enzymes, nucleotides, etc.) described elsewhere herein, in any desired combination. In certain embodiments, provided are compositions that comprise circular DNAs produced according to any of the methods of the present disclosure.

Any of the compositions of the present disclosure may be present in a container. Suitable containers include, but are not limited to, tubes, vials, plates (e.g., a 96- or other-well plate), wells of a microfluidic device, a cartridge for a sample analysis system, and/or the like.

According to some embodiments, a composition of the present disclosure comprises target nucleic acids produced according to any of the methods of the present disclosure, and/or any desired combination of reagents (e.g., primers, enzymes, nucleotides, etc.) present in a liquid medium. The liquid medium may be an aqueous liquid medium, such as water, a buffered solution, and the like. One or more additives such as a salt (e.g., NaCl, MgCl2, KCl, MgSO4), a buffering agent (a Tris buffer, N-(2-Hydroxyethyl)-piperazine-N′-(2-ethanesulfonic acid) (HEPES), 2-(N-Morpholino)-ethanesulfonic acid (MES), 2-(N-Morpholino)-ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

In some embodiments, a composition of the present disclosure is a lyophilized composition. A lyoprotectant may be included in such compositions in order to protect nucleic acids against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM. In certain aspects, a composition of the present disclosure is in a liquid form reconstituted from a lyophilized form. An example procedure for reconstituting a lyophilized composition is to add back a volume of pure water (typically equivalent to the volume removed during lyophilization); however solutions comprising buffering agents, antibacterial agents, and/or the like, may be used for reconstitution.

### Kits

Aspects of the present disclosure further include kits. In certain embodiments, a kit of the present disclosure includes any of the reagents (e.g., nucleic acids, primers, enzymes, nucleotides, etc.) described elsewhere herein, in any desired combination, and instructions for using the reagents to produce circular nucleic acids, concatemers via RCA, and/or the like in accordance with the methods of the present disclosure. The kits of the present disclosure may further include reagents and/or instructions for downstream analysis (e.g., sequencing).

According to some embodiments, a kit of the present disclosure comprises forward and reverse primers each comprising a 5′ phosphate group, a 5′ overhang region, a 3′ hybridization region that hybridizes to a target nucleic acid, and a uracil disposed between the 5′ overhang region and the 3′ hybridization region. Such a kit may further comprise a 5′ phosphorylated adapter nucleic acid adapted to: ligate to the 3′ end of a first strand of an amplicon pair via the 5′ phosphate group of the adapter nucleic acid; and ligate to a phosphorylated 5′ overhang of the second strand of the amplicon pair via the 3′ end of the adapter nucleic acid. Such kits may comprise instructions for producing a circular DNA using the forward and reverse primers and, if present, a 5′ phosphorylated adapter nucleic acid. Such kits may further comprise a ligase (e.g., a T4 ligase) that finds use in ligating a 5′ phosphorylated adapter nucleic acid to amplicon pairs produced by amplification of a target nucleic acid using the forward and reverse primers.

In certain embodiments, a kit of the present disclosure comprises forward and reverse primers each comprising first and second stem regions complementary to each other and separated by a linker region, and a 3′ hybridization region that hybridizes to a target nucleic acid. According to some embodiments, the forward primer, the reverse primer, or both, comprise one or more exonuclease resistance features present within the second stem region, the 3′ hybridization region, or both. In certain embodiments, the exonuclease resistance features comprise phosphorothioate linkages. Such kits may further comprise instructions for producing circular DNAs using the forward and reverse primers.

Any of the kits of the present disclosure may further comprise one or more RCA primers (e.g., any of the RCA primers described elsewhere herein), one or more second strand synthesis primers, an exonuclease, a polymerase (e.g., for target nucleic acid amplification and/or a fill-in reaction), a ligase, buffers, cofactors, and/or any other reagents that find use in performing the methods of the present disclosure.

Components of the kits may be present in separate containers, or multiple components may be present in a single container. For example, the forward and reverse primers may be present in separate containers or a single container. A suitable container includes a single tube (e.g., vial), one or more wells of a plate (e.g., a 96-well plate, a 384-well plate, etc.), or the like.

Instructions included in a kit of the present disclosure may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments.
1. A method for producing circular deoxyribonucleic acids (DNAs), the method comprising:
   amplifying a target nucleic acid using a forward primer and a reverse primer, wherein the forward and reverse primers each comprise a 5′ phosphate group, a 5′ overhang region, a 3′ hybridization region that hybridizes to the target nucleic acid, and a uracil disposed between the 5′ overhang region and the 3′ hybridization region, and wherein the amplifying produces amplicon pairs, each amplicon pair comprising first and second ends each comprising a phosphorylated 5′ overhang;
   ligating a 5′ phosphorylated adapter nucleic acid to the first and second ends of the amplicon pairs, wherein at each of the first and second ends of an amplicon pair, the 5′ end of the adapter nucleic acid is ligated to the 3′ end of a first strand of the amplicon pair, and the 3′ end of the adapter nucleic acid is ligated to the phosphorylated 5′ overhang of the second strand of the amplicon pair, to produce a circular DNA.
2. A method for producing circular deoxyribonucleic acids (DNAs), the method comprising:
   amplifying a target nucleic acid using a forward primer and a reverse primer, wherein the forward and reverse primers each comprise first and second stem regions complementary to each other and separated by a linker region, and a 3′ hybridization region that hybridizes to the target nucleic acid, wherein the amplifying produces amplicon pairs, each amplicon pair comprising first and second strands, and wherein each end of each of the first and second strands comprises the first stem region, the linker region and the second stem region; and
   combining the amplicon pairs with an exonuclease, a DNA polymerase and a DNA ligase under conditions in which, at each end of the amplicon pairs:
      the exonuclease removes the first stem region, the linker region and at least a portion of the second stem region from the 3′ end of the first strand of an amplicon pair,
      the first stem region and the second stem region of the second strand of the amplicon pair hybridize to each other to form a stem loop structure,
      the DNA polymerase fills in a gap between the 3′ end of the first strand of the amplicon pair and the 5′ end of the second strand of the amplicon pair, and
      the DNA ligase ligates the 3′ end of the first strand of the amplicon pair to the 5′ end of the second strand of the amplicon pair, to produce a circular DNA.
3. The method according to embodiment 2, wherein the conditions comprise a hybridization temperature at which the first and second stem regions hybridize to each other to form the stem loop structure.
4. The method according to embodiment 3, wherein the hybridization temperature is from 45° C. to 55° C.
5. The method according to embodiment 4, wherein the hybridization temperature is about 50° C.
6. The method according to any one of embodiments 2 to 5, wherein the forward primer, the reverse primer, or both, comprise one or more exonuclease resistance features present within the second stem region, the 3′ hybridization region, or both.
7. The method according to embodiment 6, wherein the exonuclease resistance features comprise phosphorothioate linkages.
8. The method according to any one of embodiments 1 to 7, wherein the target nucleic acid is a target DNA.
9. The method according to embodiment 8, wherein the target DNA is a target genomic DNA.
10. The method according to embodiment 9, wherein the target DNA is a target complementary DNA (cDNA).
11. The method according to any one of embodiments 1 to 7, wherein the target nucleic acid is a target ribonucleic acid (RNA).
12. The method according to any one of embodiments 1 to 11, further comprising performing rolling circle amplification (RCA) using the circular DNAs as templates, wherein the RCA produces concatemers comprising repeating segments each comprising a target nucleic acid sequence.
13. The method according to embodiment 12, wherein the circular DNAs were produced according to the method of embodiment 1, and wherein the RCA uses a primer that anneals to the adapter nucleic acid.
14. The method according to embodiment 12, wherein the circular DNAs were produced according to the method of embodiment 2, and wherein the RCA uses a primer that anneals to the loop portion of the stem loop structure.
15. The method according to any one of embodiments 12 to 14, further comprising sequencing the concatemers using a nanopore sequencing device.
16. The method according to embodiment 15, wherein the sequencing comprises producing a consensus sequence of the target nucleic acid sequence.
17. A kit comprising:
   forward and reverse primers each comprising a 5′ phosphate group, a 5′ overhang region, a 3′ hybridization region that hybridizes to a target nucleic acid, and a uracil disposed between the 5′ overhang region and the 3′ hybridization region; and
   instructions for producing circular DNAs using the forward and reverse primers.
18. The kit of embodiment 17, further comprising a 5′ phosphorylated adapter nucleic acid adapted to: ligate to the 3′ end of a first strand of an amplicon pair via the 5′ phosphate group of the adapter nucleic acid; and ligate to a phosphorylated 5′ overhang of the second strand of the amplicon pair via the 3′ end of the adapter nucleic acid.
19. A kit comprising:
   forward and reverse primers each comprising first and second stem regions complementary to each other and separated by a linker region, and a 3′ hybridization region that hybridizes to a target nucleic acid; and
   instructions for producing circular DNAs using the forward and reverse primers.
20. The kit of embodiment 19, wherein the forward primer, the reverse primer, or both, comprise one or more exonuclease resistance features present within the second stem region, the 3′ hybridization region, or both.
21. The kit of embodiment 20, wherein the exonuclease resistance features comprise phosphorothioate linkages.
22. The kit of any one of embodiments 17 to 21, further comprising an RCA primer.
23. The kit of any one of embodiments 17 to 22, wherein one or more primers present in the kit comprise a sequencing adapter for a nanopore sequencing device.

The following examples are offered by way of illustration and not by way of limitation.

### EXPERIMENTAL

### Example 1—Production of Circular DNAs, Rolling Circle Amplification and Nanopore Sequencing

Described herein is the production of circular DNAs according to the first and second aspects of the methods of the present disclosure, as well as subsequent rolling circle amplification (RCA) and sequencing of the concatemers resulting from the RCA.

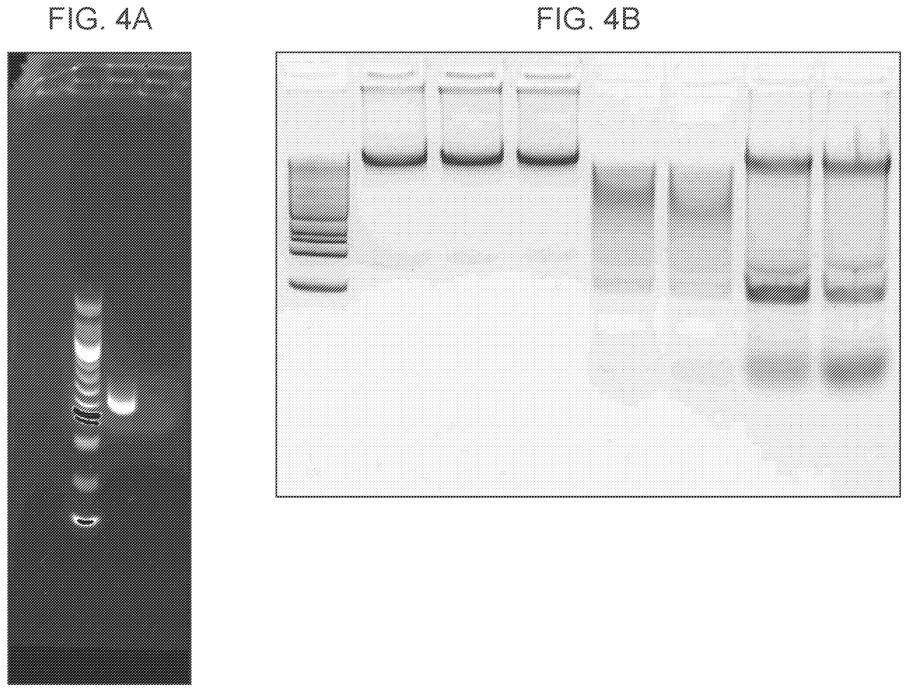

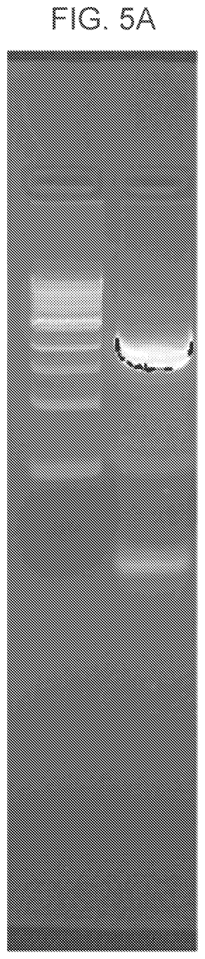

FIG. 4A shows an electrophoresis gel image showing the expected amplicon size from an amplification reaction according to methods of the first aspect of the present disclosure schematically illustrated in FIG. 1. In this example, a Q5 PCR reaction was performed using primers containing a uracil, at ˜600 bp in relation to the NEB 100 bp DNA Ladder. The PCR included 32 cycles of: denaturation at 98° C. (10 s), annealing at 58° C. (20 s), and extension at 72° C. (2 minutes). The right-most lane=NTC (no template control). The use a limiting concentration of primers resulted in a theoretical maximum number of amplicons and eliminated the need for quantification.

FIG. 4B shows an electrophoresis gel image showing high molecular weight DNA from the successful Phi29 colling circle amplification of circular generated products using short oligonucleotides. Products were generated as follows: an amplicon containing phosphorylated overhangs due to the inclusion of uracil in the primer sequence (FIG. 4A) was incubated with hairpin oligonucleotides and T4 ligase with the control having no hairpin oligonucleotides added; (2) DNA was then purified using AMPure XP beads (Beckman Coulter) and incubated with Exonuclease VIII truncated and Exonuclease I to degrade all linear DNA; (3) rolling circle amplification was then performed using phi29 polymerase, a 6-nucleotide long oligonucleotide complementary to the loop structure and a 6-nucleotide long oligonucleotide reverse complementary to the stem of the hairpin oligonucleotide, where both oligonucleotides contained phosphorothioate links at the 5′-end to prevent degradation by phi29; and (4) a T7 endonuclease treatment was performed to resolve branching. Lane 1=NEB 1 kb Extend DNA Ladder, Lane 5=negative control T7 endonuclease treatment, Lane 6=T7 endonuclease treatment.

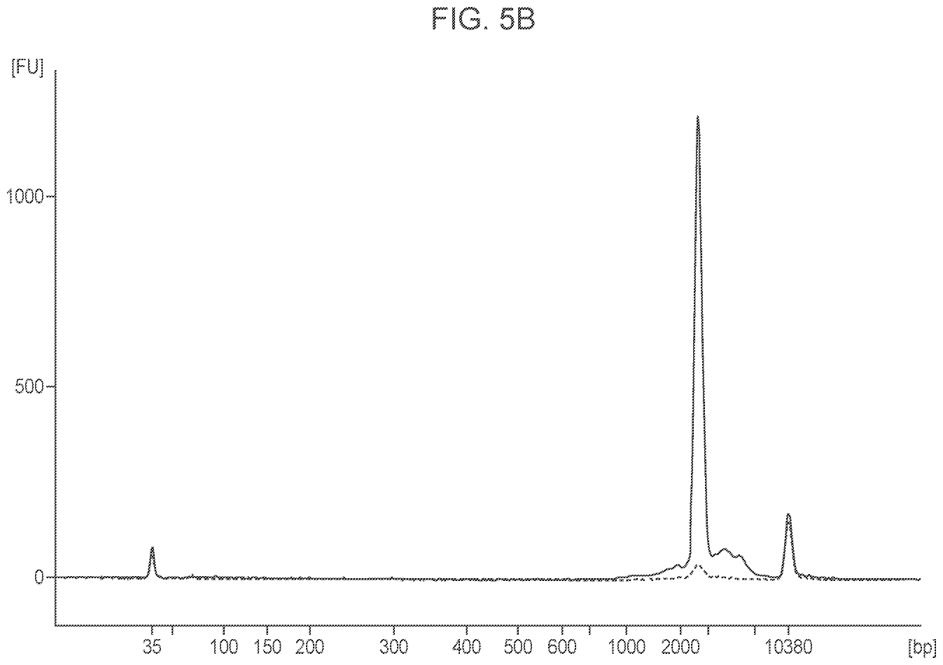

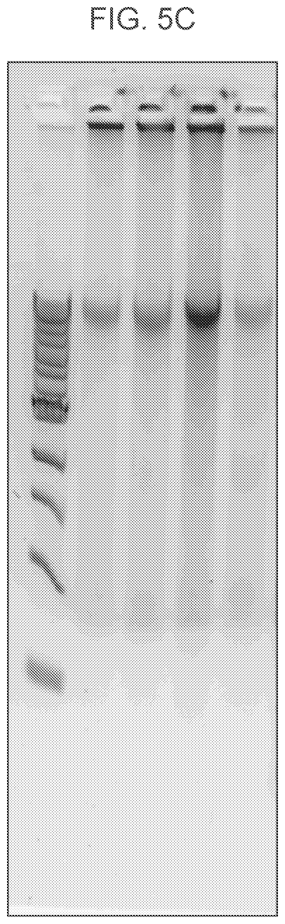

FIG. 5A shows an electrophoresis gel image showing the expected amplicon size from an amplification reaction according to methods of the second aspect of the present disclosure schematically illustrated in FIG. 2. In this example, a Q5 PCR reaction was performed using primers containing a hairpin formation sequence (as shown in FIG. 2) at ˜2.3 kb in relation to the NEB 1 kb Extend DNA Ladder. The use a limiting concentration of primers resulted in a theoretical maximum number of amplicons and eliminated the need for quantification.

FIG. 5B shows a bioanalyzer DNA electropherogram showing a large peak at ˜2.3 kb for the sample and a relatively minute peak for the control as expected. Samples were processed as follows: (1) an amplicon containing a sequence that forms a hairpin at ≤50° C. but not at the temperature above this (i.e., at the annealing temperature for the PCR reaction to generate the amplicons, there is no hairpin and the oligonucleotide is linear; FIG. 5A) was incubated with a mixture of T7 exonuclease, DNA ligase and DNA polymerase, resulting in the formation of circular DNA; and (2) the circular DNA was incubated with Exonuclease VIII truncated and Exonuclease I to degrade all linear DNA, showing that the circularization was successful (red line). FIG. 5C shows RCA reactions using the generated circular DNA as template, Phi29 polymerase, and different combinations of short oligonucleotides (lanes 2, 3 and 5) and random hexamers (lane 4) showing large (48.5 kb) DNA fragments and large (likely) hyperbranched fragments in the wells. Lane 1=NEB 1 kb Extend DNA Ladder.

For sequencing, the Oxford Nanopore Technologies transposase library preparation kit was used to fragment and attach sequencing adapters to the RCA products. The adapted products were sequenced on the Oxford Nanopore Technologies Flongle Flow Cell (R9.4.1). The results demonstrated the successfully creation of concatenated products of the desired amplicons, with some containing up to 100 copies of the original amplicon (even after the fragmentation performed for this test). The mean number of concatenated copies of the amplicon sequence were 7.2 (SD=6.9) and 4.5 (SD=3) for the circularization methods according to the first and second aspects, respectively.

### Example 2—Circularization of Rv0678 for Genotypic Bedaquiline Resistance Testing of Mycobacterium tuberculosis

Circular DNA offers benefits over linear DNA in diagnostic and field assays, including resistance to most exonucleases—which catalyze the removal of nucleotides from the free ends of single-, or double-stranded DNA by hydrolyzing phosphodiester bonds and the ability to act as a template for rolling circle amplification—which is an isothermal process of unidirectional nucleic acid replication resulting in concatenated copies of the circular template.

Rolling circle amplification (RCA) was first developed as a method in the mid-1990s. An application of RCA is to generate long, single-stranded concatemers of DNA as the template for long-read sequencing as with Oxford Nanopore Technologies (ONT) platforms, where it allows for error correction by taking a consensus of the de-concatenated sequence. Sequencing of longer DNA strands also improves the output from the sequencer, as short reads exhaust nanopores more quickly. ONT sequencing is highly sought after as this technology is portable and requires little infrastructure, allowing for its utilization in the field, for example, in the characterization of unique ecological niches. It also has a potentially pioneering impact on diagnostics and clinical practice, for example, in the point-of-care genotypic drug susceptibility testing of pathogens, such as Mycobacterium tuberculosis.

Current methods of generating circular DNA are lengthy, inefficient, highly dependent on the length and sequence of DNA, and can result in unwanted chimeras. The circularization of single-stranded DNA is the most common and is used for the generation of templates for short-read sequencing in Beijing Genomics Institute (BGI)'s nanoball technology. A second approach is to clone a fragment into a vector, resulting in circular DNA; however, this will always result in the presence of the vector backbone. A third approach is to ligate dumbbell (hairpin) oligos to linear dsDNA as utilized by Pacific Biosciences in their Single Molecule, Real-Time (SMRT) sequencing system. The pseudo-circularized DNA then serves as an RCA template.

Described herein are improved methods for generating PCR (or similar techniques, including strand displacement and recombinase polymerase amplification) targeted circular DNA. As proof-of-principle, the present example focuses on generating a ˜700 bp amplicon of rv0678, the high GC content (65%) gene implicated in bedaquiline resistance in M. tuberculosis, the causative agent of tuberculosis. Several methods are presented, including using splints, a Gibson cloning-based approach for self-circularization, as wells as the new methods for generating pseudo-circular DNA.

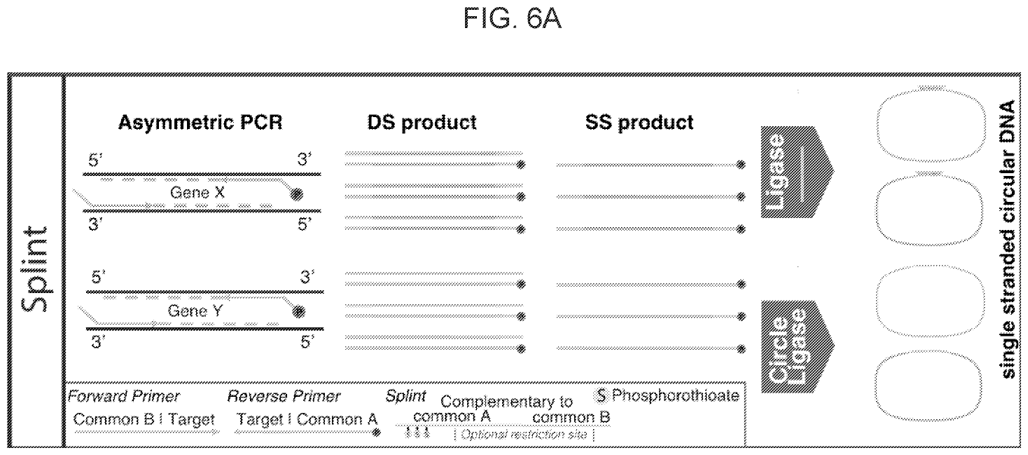

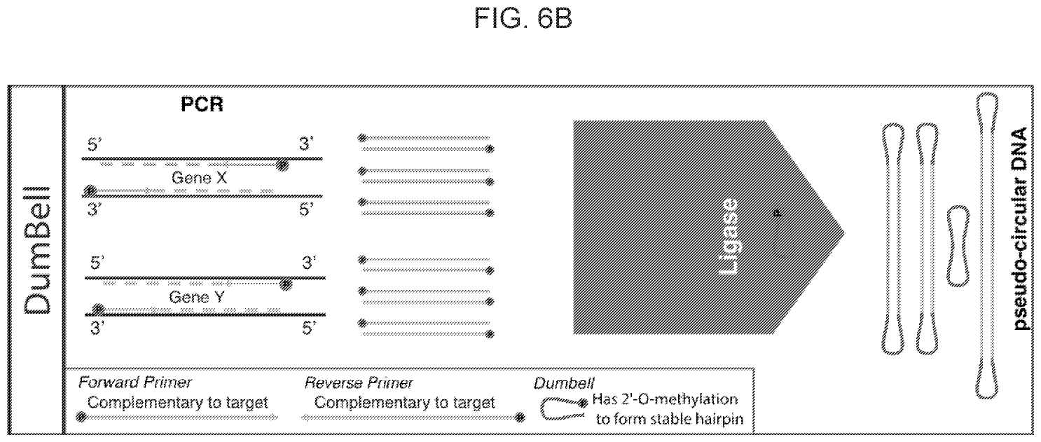

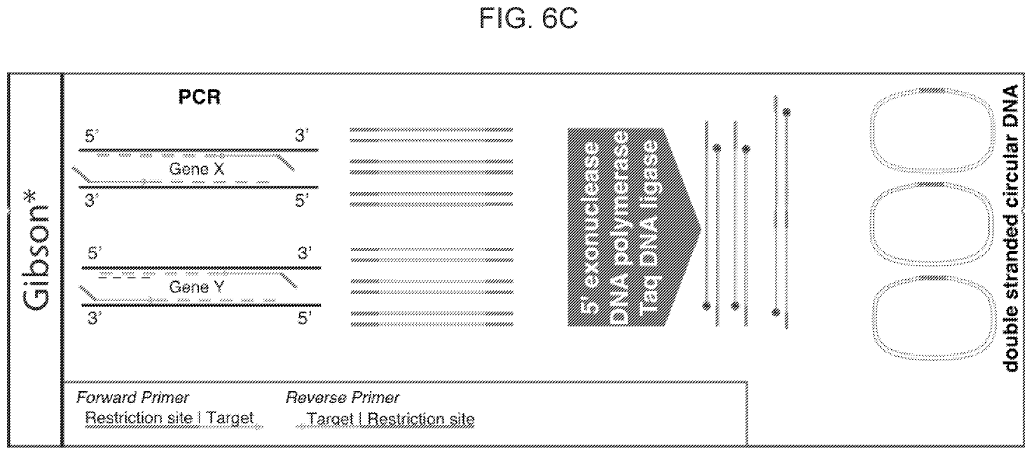

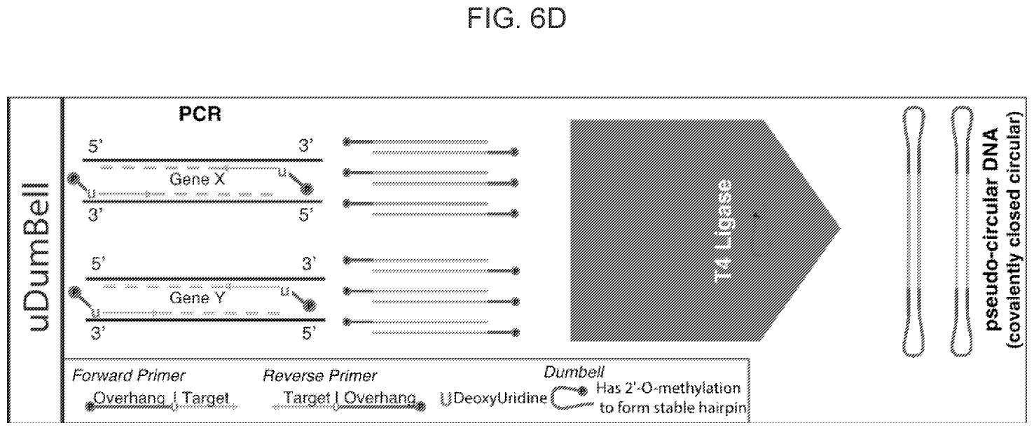

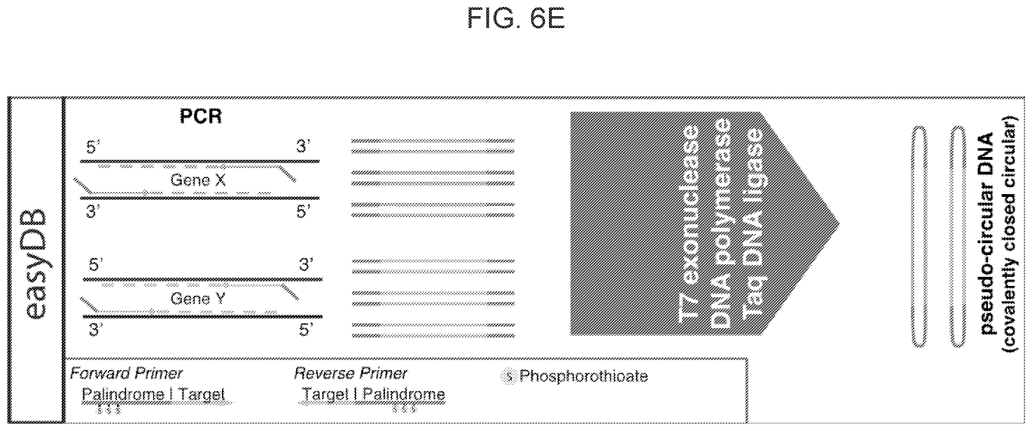

In this example, ˜700 nt amplicons spanning the rv0678 gene from M. tuberculosis H37Rv DNA were generated as a template for the various methods using the primer set “initial amplicon generation” (Table 1—Methods). The different amplicons were then generated utilizing the remainder of the primer pairs in Table 1. Following the various incubations (FIG. 6), specific exonucleases were used to eliminate non-circular or non-pseudocircular DNA.

### Single-Stranded Circular DNA

Previously utilized methods for single-stranded DNA circularization include using a specialized ligase, CircleLigase. CircleLigase is most often used on short (<200 nucleoties) strands. Here, the generation of single-stranded DNA of a large, high GC-content amplicon was simplified, and self-circularization was attempted using CircleLigase and an oligonucleotide splint, and common ligases.

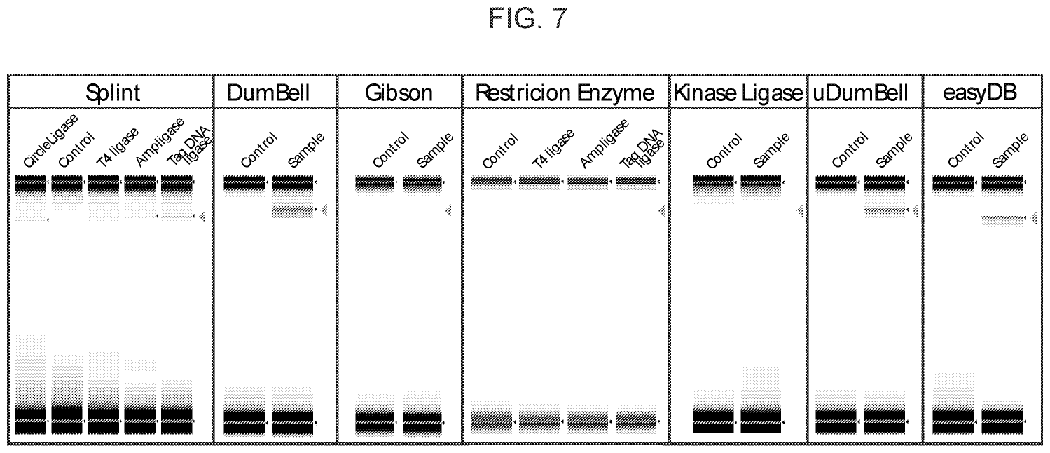

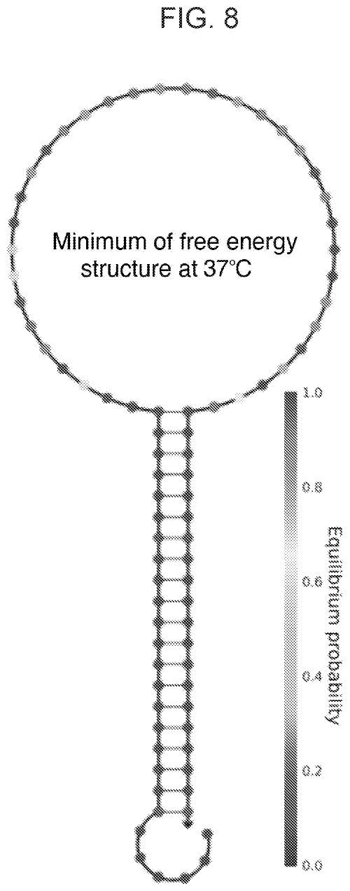

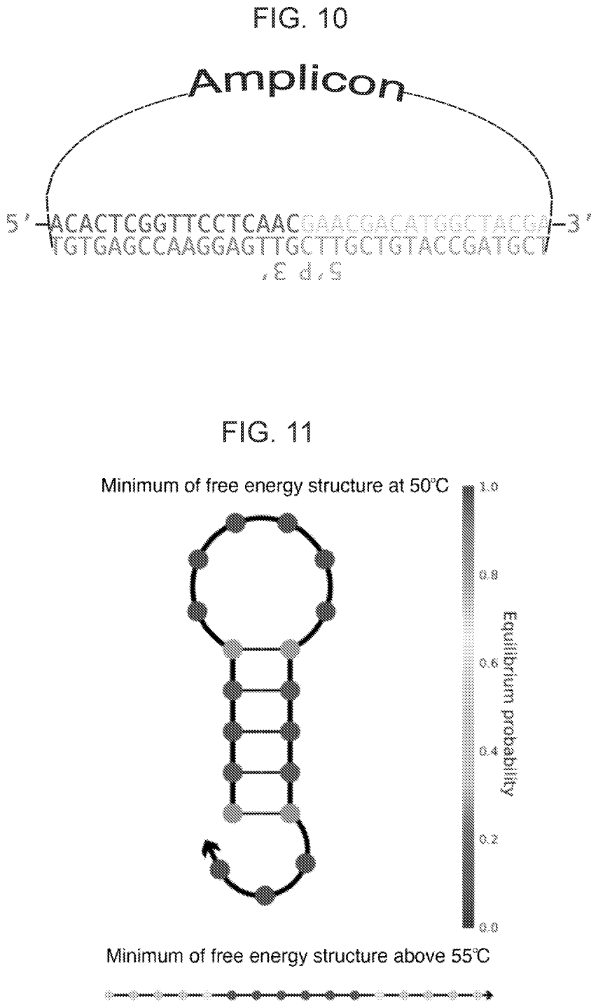

Self-circularization of phosphorylated single-stranded DNA using CircleLigase was successful; however, it appears inefficient (FIG. 7—Splint). Circularization using a splint complementary (FIG. 10) to the two ends of the single-stranded DNA and incubation with either T4 ligase, Ampligase, or Taq DNA ligase similarly resulted in low concentrations of single-stranded circular DNA, with T4 ligase performing the poorest.

### Double-Stranded Circular DNA

Commonly utilized methods of double-stranded DNA circularization are used in cloning, where a double-stranded fragment is cloned into a large double-stranded backbone. If the large backbone is not dephosphorylated, it will often self-recircularize. Attempted here was self-circularization of a large, high GC content amplicon.

Amplicons with fifteen complementary bases at each end were generated for self-circularization using the Gibson cloning, and kinase, ligase treatment reactions. The Gibson reaction resulted in no detectable circular DNA, while the kinase-ligase treatment did not result in a single circularized amplicon, but it did produce large, concatenated circles. Also generated were amplicons with commentary Xball sites at the ends and digested the amplicons accordingly, which were incubated with T4 ligase, Ampligase, or Taq DNA ligase. In all cases, double-stranded circular DNA was not observed. Attempts were made using the restriction sites NdeI and KpnI, and a twenty-seven-base pair insert with no success.

### Double-Stranded Pseudo-Circular DNA

The ligation of dumbbell (hairpin) oligos to linear dsDNA is used by PacBio to create double-stranded pseudo-circular DNA. This method was attempted, resulting in two streamlined variations to produce double-stranded pseudo-circular DNA of a large, high GC content amplicon.

Ligation of DumBells—single-stranded, phosphorylated dumbbell (hairpin) DNA—to double-stranded phosphorylated DNA following DNA purification was successful but did result in amplicon concatemerization and circular DNA formation of only dumbbell (hairpin) DNA (FIG. 7—DumBell).

Including deoxyUridine in the PCR primer sequences causes Q5 and other high-fidelity polymerases to arrest elongation. This results in overhangs that were successfully ligated to a complementary dumbbell (hairpin) D structure (FIG. 7—uDumBell). The deoxyUridine reduced the PCR product by approximately two-thirds, but this was ameliorated by increasing the Q5 DNA polymerase concentration three-fold.

Primers were designed with a tail sequence that forms a six-nucleotide hairpin at temperature<55° C., but not >55° C. (FIG. 11). These primers contain six phosphorothioate bonds starting at the complementary region to inhibit exonuclease T7 activity. The primers successfully amplified the target and, following incubation with a mixture of T7 exonuclease, DNA polymerase, and Taq DNA ligase, pseudo-circular double-stranded DNA formed (FIG. 7—easyDB).

Also made was pseudo-circular DNA using TeIN protelomerase, which cuts dsDNA at a 56 bp recognition sequence and leaves covalently closed ends at the cleavage site.

To summarize, demonstrated herein are several methods, including using splints, a Gibson cloning-based approach for self-circularization, and novel methods for generating pseudo-circular DNA from a ˜700 bp amplicon of rv0678, the high GC content (65%) gene implicated in bedaquiline resistance in M tuberculosis, the causative agent of tuberculosis. This circular DNA can be used as a template for RCA followed by long read sequencing, allowing for the error correction of sequence data, and improving confidence in the resistance determination. The protection of circular DNA from degradation has applications in DNA vaccines, where DNA must be delivered into cells and make its way into the nucleus to assert its effects. Linear DNA with free ends is more recombinogenic27 and has lower transfection efficiencies and expression than DNA minicircles28 (dsDNA supercoiled circles containing only the genes of interest). The behavior of pseudo-circular DNA, however, is unknown. Pseudo-circular DNA is linear, double-stranded DNA with covalently closed (hairpin) ends and, unlike plasmids and minicircles, has no lower size limit. For these reasons, pseudo-circular DNA is expected to have applications in transgenics and DNA vaccines.

### Methods for Example 2

### Initial Amplicon Generation

The initial ˜700 bp amplicon were generated using the primer set “initial amplicon generation” (Table 1) from genomic M. tuberculosis H37Rv DNA with Q5 polymerase (NEB, USA) according to the manufacturer's instructions. The thermocycling was done as follows: initial denaturation at 98° C. for 30 seconds, 34 cycles of 98° C., 62° C., and 72° C. for 10, 10, and 20 seconds respectively. Amplicons were purified using 0.8× Agencourt AMPureXP beads (BD, USA) according to the manufacturer's instructions. This template was then used, with the primers in Table 1 to generate the remaining amplicons using the same procedure.

TABLE 1
Primer sequences. An asterisk between nucleotides denotes a phosphorothioate bond.

| Method | Forward Primer Sequence | Reverse Primer Sequence |
|---|---|---|
| Initial amplicon | TTTCTGTTGGTGCTGATATTGCct | ACTTGCCTGTCGCTCTATCTTCac |
| generation | ggtgacgcataccgaacg (SEQ | ctcggtcagattgcgagg (SEQ |
| | ID NO: 1) | ID NO: 19) |
| Splint | /5Phos/GAACGACATGGCTACGA | TGTGAGCCAAGGAGTTGACTTGCC |
| | TTTCTGTTGGTGCTGATATTGC | TGTCGCTCTATCTTC (SEQ ID |
| | (SEQ ID NO: 2) | NO: 9) |
| DumBell | /5Phos/TTTCTGTTGGTGCTGAT | /5Phos/ACTTGCCTGTCGCTCTA |
| | ATTGC (SEQ ID NO: 3) | TCTTC (SEQ ID NO: 10) |
| Gibson cloning | GGCGGCGACCTGTCGTTTCTGTTG | CGACAGGTCGCCGCCACTTGCCTG |
| | GTGCTGATATTGC (SEQ ID | TCGCTCTATCTTC (SEQ ID |
| | NO: 4) | NO: 11) |
| Restriction enzyme | AAAATCTAGATTTCTGTTGGTGCT | AAAAAGATCTACTTGCCTGTCGCT |
| cloning | GATATTGC (SEQ ID NO: 5) | CTATCTTC (SEQ ID NO: 12) |
| TeIN | TATCAGCACACAATTGCCCATTAT | ATAGTCGTGTGTTATCAGGTAATA |
| | ACGCGCGTATAATGGACTATTGTG | TGCGCGCATATTACCCGTTAACAC |
| | TGCTGATATTTCTGTTGGTGCTGA | ACGACTATACTTGCCTGTCGCTCT |
| | TATTGC (SEQ ID NO: 6) | ATCTTC (SEQ ID NO: 13) |
| uDumBell | /5Phos/GUCTATTTTCTGTTGGT | /5Phos/GUCTATACTTGCCTGTC |
| | GCTGATATTGC (SEQ ID | GCTCTATCTTC (SEQ ID |
| | NO: 7) | NO: 14) |
| eDumBell | GGCGTCTCAAAACGCCCGT*T*T* | GGCGTCTCAAAACGCCCGT*A*C* |
| | T*C*T*GTTGGTGCTGATATTGC | T*T*GCCTGTCGCTCTATCTTC |
| | (SEQ ID NO: 8) | (SEQ ID NO: 15) |

### DNA Circularization Procedures

### Splint

Two micrograms of amplicon was digested with Lambda Exonuclease (NEB, USA) in a 30 ul reaction at 37° C. for 30 minutes according to the manufacturer's instructions. The reaction was stopped by adding EDTA to 20 mM and incubating at 75° C. for 10 minutes. Following a 1.8× AMPureXP bead cleanup, T4 Polynucleotide Kinase (NEB, USA) was used to phosphorylate the 5′-end according to the manufacturer's instructions. A 1.8×AMPureXP bead cleanup was done, and 100 ng of the resulting single-stranded material was used to generate single-stranded circular DNA described as follows.

CircleLigase. One hundred units of CircleLigase was used in a 20 ul reaction set up according to the manufacturer's instructions. The reaction was incubated at 60° C. for four hours, followed by the inactivation of the enzyme at 80° C. for 10 minutes.

Splint ligation. Two nanomolar of the splint (ACACTCGGTTCCTCAACGAACGACATGGCTACGA; SEQ ID NO:16; FIG. 10) was incubated with the amplicons in a reaction without the ligase or buffer addition, at 80° C. for 5 minutes and slowly cooled to 4° C. using a ramp rate of 0.1 C/s. Either T4 ligase (NEB, USA), Ampligase (Biosearch Technologies, UK), or DNA ligase (NEB, USA) and the corresponding buffer were added and incubated as follows. For the T4 ligase reaction, 22° C., 15° C., 4° C. for 30, 120, and 120 minutes; for the Ampligase reaction, 60° C., 55° C., 45° C. for 10, 10, and 120 minutes; and for the Taq DNA ligase reaction, 70° C., 65° C., gradient with ramp rate of 0.1 C/s to 60° C. for 10, 10, and 90 minutes.

DumBell and uDumBell

The dumbbell (hairpin) adapters (/5Phos/CGAGACAGTAGAAGACCATGAACAAGCAGCACACGATAAACTAGACACCCTACTGTCTCG (SEQ ID NO:17) and /5Phos/ATAGACCGAGACAGTAGAAGACCATGAACAAGCAGCACACGATAAACTAGACACCCTACTGTC TCG (SEQ ID NO: 18)) were prepared by incubating at 80° C. followed by cooling to room temperature over 30 minutes. Two hundred nanograms of the amplicon was incubated with 1 μm of the adapter with T4 ligase in a 30 ul reaction at 22° C., 15° C., 4° C. for 30, 120, and 120 minutes and inactivated at 65° C. for 5 minutes.

### Gibson

Four hundred nanograms of the amplicon was incubated with NEBuilder HiFi DNA Assembly Master Mix (NEB, USA) in a 20 ul reaction at 50° C. for 60 minutes.

### Restriction Enzyme

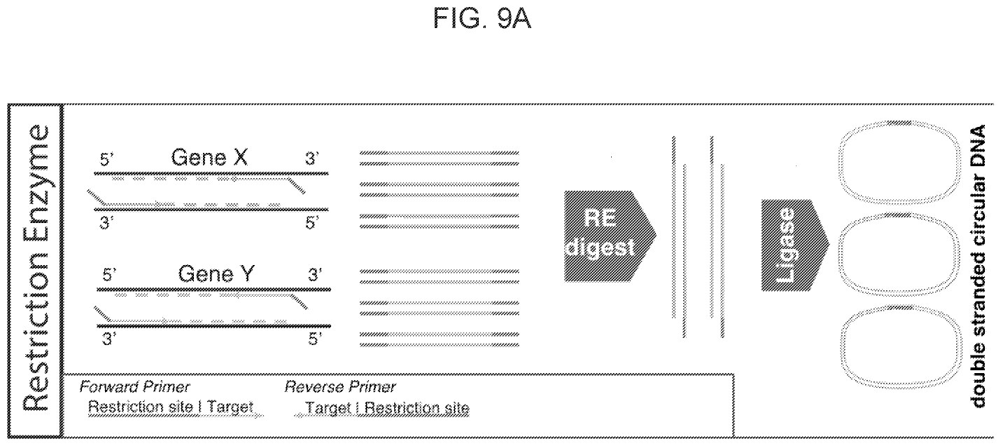

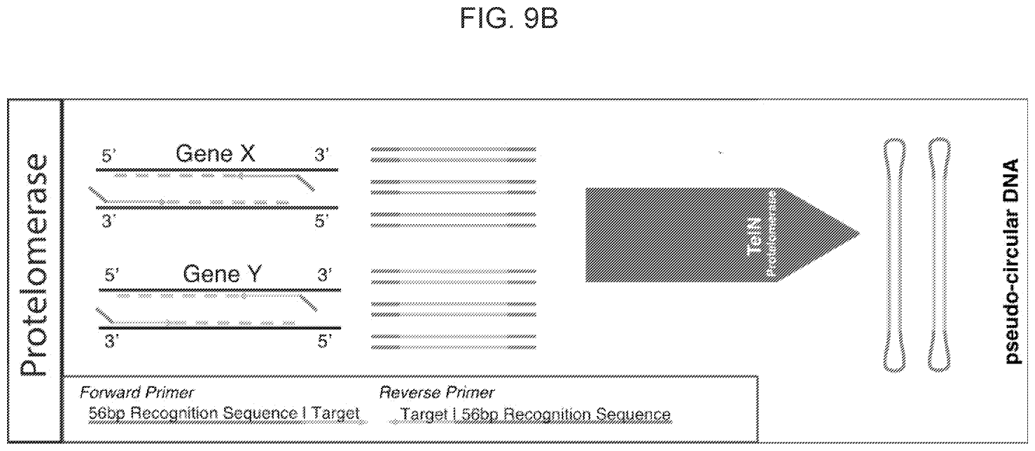

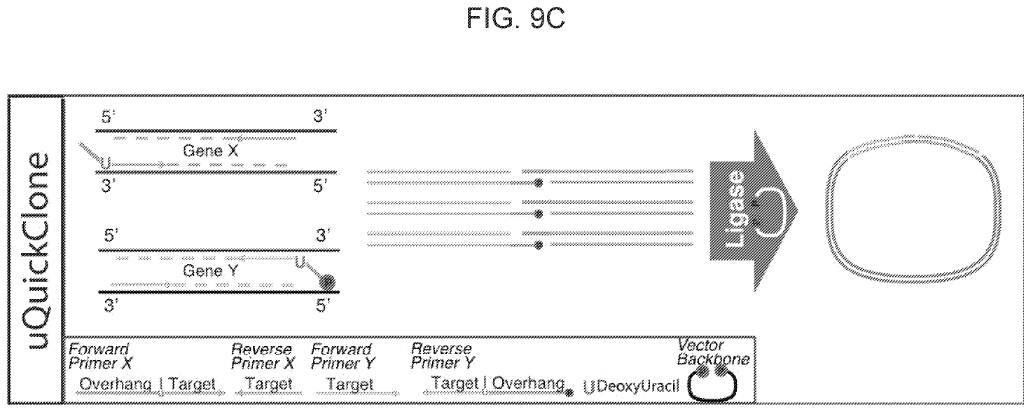

Eight hundred nanograms of the amplicon was digested with Xball (NEB, USA) at 37° C. for 30 minutes, followed by a 1×AMPureXP bead cleanup. Two hundred nanograms of this was incubated with the amplicons in a reaction without the ligase or buffer addition, at 80° C. for 5 minutes and slowly cooled to 4° C. using a ramp rate of 0.1 C/s. Buffer and either T4 ligase, Ampligase, or Taq DNA ligase were added and incubated as described in Splint ligation (FIG. 9).

### Kinase-Ligase

Four hundred nanograms of the amplicon was incubated with KLD Mix (NEB, USA) in a 20 ul reaction at room temperature for 30 minutes.

### TeIN

One hundred nanograms of the amplicon was incubated with 10 U of TeIN Protelomerase (NEB, USA) according to the manufacturer's instructions at 30° C. for 30 minutes, followed by inactivation at 75° C. for 5 minutes (FIG. 9).

easyDB

Four-hundred nanograms of the amplicon was incubated with easyDumBell (easyDB) buffer containing 0.05 U of T7 exonuclease, 0.03 U of Phusion polymerase, and 53 U Taq DNA ligase, 110 mMmM Tris-HCl pH 7.5, 15 mM MgCl2, 0.4 mM dGTP, 0.1 mM dATP, 0.1 mM dTTP, 0.1 mM dCTP, 4 mM DTT, 4% PEG 8000, and 0.2 mM NAD+(all sourced from NEB, USA) in a 20 ul reaction at 50° C. for 60 minutes.

### Exonuclease Treatment to Remove Non-Circular and Non-Pseudo Circular DNA

Twenty microliter reactions containing 10 U Exonuclease VIII truncated (NEB, USA) were set up according to the manufacturer's instructions and incubated at 37° C. for 30 minutes. The reaction was inactivated by adding 24 mM EDTA and incubating at 70° C. for 30 minutes. A 1.8×AMPureXP bead cleanup was then done. For the easyDB method, 50 U of Exonuclease Ill was also included in the reaction, and the reaction was incubated at 37° C. for 1 hour before enzyme inactivation.

### TapeStation

Samples were run on the Agilent TapeStation (Agilent, USA) using the D1000 kit according to the manufacturer's instructions.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A method for producing circular deoxyribonucleic acids (DNAs), the method comprising:
amplifying a target nucleic acid using a forward primer and a reverse primer, wherein the forward and reverse primers each comprise a 5′ phosphate group, a 5′ overhang region, a 3′ hybridization region that hybridizes to the target nucleic acid, and a uracil disposed between the 5′ overhang region and the 3′ hybridization region, and wherein the amplifying produces amplicon pairs, each amplicon pair comprising first and second ends each comprising a phosphorylated 5′ overhang;
ligating a 5′ phosphorylated adapter nucleic acid to the first and second ends of the amplicon pairs, wherein at each of the first and second ends of an amplicon pair, the 5′ end of the adapter nucleic acid is ligated to the 3′ end of a first strand of the amplicon pair, and the 3′ end of the adapter nucleic acid is ligated to the phosphorylated 5′ overhang of the second strand of the amplicon pair, to produce a circular DNA.

2. A method for producing circular deoxyribonucleic acids (DNAs), the method comprising:
amplifying a target nucleic acid using a forward primer and a reverse primer, wherein the forward and reverse primers each comprise first and second stem regions complementary to each other and separated by a linker region, and a 3′ hybridization region that hybridizes to the target nucleic acid, wherein the amplifying produces amplicon pairs, each amplicon pair comprising first and second strands, and wherein each end of each of the first and second strands comprises the first stem region, the linker region and the second stem region; and
combining the amplicon pairs with an exonuclease, a DNA polymerase and a DNA ligase under conditions in which, at each end of the amplicon pairs:
the exonuclease removes the first stem region, the linker region and at least a portion of the second stem region from the 3′ end of the first strand of an amplicon pair,
the first stem region and the second stem region of the second strand of the amplicon pair hybridize to each other to form a stem loop structure,
the DNA polymerase fills in a gap between the 3′ end of the first strand of the amplicon pair and the 5′ end of the second strand of the amplicon pair, and
the DNA ligase ligates the 3′ end of the first strand of the amplicon pair to the 5′ end of the second strand of the amplicon pair, to produce a circular DNA.

3. The method according to claim 2, wherein the conditions comprise a hybridization temperature at which the first and second stem regions hybridize to each other to form the stem loop structure.

4. The method according to claim 3, wherein the hybridization temperature is from 45° C. to 55° C.

5. The method according to claim 4, wherein the hybridization temperature is about 50° C.

6. The method according to claim 2, wherein the forward primer, the reverse primer, or both, comprise one or more exonuclease resistance features present within the second stem region, the 3′ hybridization region, or both.

7. The method according to claim 6, wherein the exonuclease resistance features comprise phosphorothioate linkages.

8. The method according to claim 1, wherein the target nucleic acid is a target DNA.

9. The method according to claim 8, wherein the target DNA is a target genomic DNA.

10. The method according to claim 9, wherein the target DNA is a target complementary DNA (cDNA).

11. The method according to claim 1, wherein the target nucleic acid is a target ribonucleic acid (RNA).

12. The method according to claim 1, further comprising performing rolling circle amplification (RCA) using the circular DNAs as templates, wherein the RCA produces concatemers comprising repeating segments each comprising a target nucleic acid sequence.

13. The method according to claim 12, wherein the RCA uses a primer that anneals to the adapter nucleic acid.

14. The method according to claim 2, further comprising performing rolling circle amplification (RCA) using the circular DNAs as templates, wherein the RCA produces concatemers comprising repeating segments each comprising a target nucleic acid sequence, and wherein the RCA uses a primer that anneals to the loop portion of the stem loop structure.

15. The method according to claim 12, further comprising sequencing the concatemers using a nanopore sequencing device.

16. The method according to claim 15, wherein the sequencing comprises producing a consensus sequence of the target nucleic acid sequence.

17. A kit comprising:
(a) forward and reverse primers each comprising a 5′ phosphate group, a 5′ overhang region, a 3′ hybridization region that hybridizes to a target nucleic acid, and a uracil disposed between the 5′ overhang region and the 3′ hybridization region, or
(b) forward and reverse primers each comprising first and second stem regions complementary to each other and separated by a linker region, and a 3′ hybridization region that hybridizes to a target nucleic acid; and
instructions for producing circular DNAs using the forward and reverse primers of (a) or (b).

18. The kit of claim 17, comprising the forward and reverse primers of (a), further comprising a 5′ phosphorylated adapter nucleic acid adapted to: ligate to the 3′ end of a first strand of an amplicon pair via the 5′ phosphate group of the adapter nucleic acid; and ligate to a phosphorylated 5′ overhang of the second strand of the amplicon pair via the 3′ end of the adapter nucleic acid.

19. (canceled)

20. The kit of claim 17, comprising the forward and reverse primers of (b), wherein the forward primer, the reverse primer, or both, comprise one or more exonuclease resistance features present within the second stem region, the 3′ hybridization region, or both.

21. The kit of claim 20, wherein the exonuclease resistance features comprise phosphorothioate linkages.

22.-23. (canceled)
